(19) United States
(12) Patent Application Publication
(10) Pub. No.: US 2014/0135396 A1
(43) Pub. Date: May 15, 2014

(54) Treatment of Acetaminophen-Induced Liver Damage by the Administration of Modulators of Nitric Oxide

(71) Applicants: Wolfram Goessling, Chestnut Hill, MA (US); Andrew Cox, Boston, MA (US); Trista North, Newton Center, MA (US)

(72) Inventors: Wolfram Goessling, Chestnut Hill, MA (US); Andrew Cox, Boston, MA (US); Trista North, Newton Center, MA (US)

(73) Assignees: BETH ISRAEL DEACONESS MEDICAL CENTER, INC., Boston, MA (US); THE BRIGHAM AND WOMEN'S HOSPITAL, INC., Boston, MA (US)

(21) Appl. No.: 14/130,548

(22) PCT Filed: Jul. 4, 2012
(86) PCT No.: PCT/US2012/045476

§ 371 Date: Jan. 2, 2014

Related U.S. Application Data
(60) Provisional application No. 61/504,544, filed on Jul. 5, 2011.

(51) Int. Cl. A61K 31/198; A61K 31/197

(52) U.S. Cl. CPC A61K 31/198; A61K 31/197
USPC 514/562; 514/565; 514/564; 514/563

(57) ABSTRACT

The invention is directed to the treatment of patients at risk for liver damage due to the overingestion of acetaminophen by administering agents that increase hepatic levels of nitric oxide.

### CROSS REFERENCE TO RELATED APPLICATIONS

The present application claims the benefit of U.S. provisional application 61/504,544, filed on Jul. 5, 2011. This prior application is hereby incorporated by reference in its entirety.

### FIELD OF THE INVENTION

The present invention is in the field of the treatment or prevention of liver damage in patients caused by the overingestion of toxic substances. In particular, it is directed to methods of treating patients that have overingested acetaminophen by administering agents that increase hepatic levels of nitric oxide.

### BACKGROUND OF THE INVENTION

Acetaminophen (also known as paracetamol and N-acetyl-p-aminophenol (APAP)) is one of the most commonly used analgesics (Kaufman, et al., JAMA 16:287(3) 337-44 (2002)) and is available in the United States as 325-mg and 500-mg tablets, as well as in a 650-mg extended-release preparation. It is also in many over-the-counter cold and analgesic medications. Although effective at relieving pain and reducing fever, taking more than the maximum recommended amount of acetaminophen (more than about 4 grams per day for an adult) can cause severe, and sometimes fatal, liver damage (Larson, et al., Hepatology 42:1364-1372 (2005)). At present, common treatments for acute acetaminophen poisoning include the administration of N-acetylcysteine (Kanter, Am. J. Health Syst. Pharm. 63:1821-1827 (2006)); and/or activated charcoal (Buckley, et al, Clin. Tox. 37:753-757 (1999)).

Nitric oxide (NO) has a wide variety of biological effects and potential therapeutic uses. It is a vasodilator, antioxidant, anti-inflammatory, antithrombotic, modulator of skeletal muscle and myocardial contractility, and is intimately linked with insulin signaling (Levine, et al., Cardiology 122:55-68 (2012)). In the US, it has been approved by the FDA for the treatment hypoxic respiratory failure in infants (Committee on Fetus and Newborn, Pediatrics 106:344-345 (2012)). With respect to the liver, there have been studies suggesting that NO may be beneficial in the treatment of arsenic-induced toxicity in rats (Qu, et al., Chemico-Biolog. Interactions 193:88-96 (2011)) and in the prevention of ischemic reperfusion injury (Abu-Amara, et al., Liver Internat'l 32:521-543 (2012)).

Because of its many beneficial actions, agents that are capable of increasing biological levels of NO are of great therapeutic interest. These agents may take the form of compounds that donate NO molecules or agents that act metabolically to increase NO concentration in vivo. Among the most promising compounds are those that act by blocking the action of S-nitrosoglutathione reductase (GSNOR, also known as glutathione-dependent formaldehyde dehydrogenase, GS-FDH), an NADH-dependent enzyme that catalyzes the reduction of S-nitrosoglutathione (Sun, et al., Bioorg. Med. Chem. Lett. 21:5849-5853 (2011); U.S. Pat. No. 7,179,791). Effective inhibitors of this enzyme have been reported by Stamler (U.S. Pat. NO. 7,615,535; US 2010/0015121) and by Wasley et al (WO 2010/019903 and WO2010/019910).

### SUMMARY DESCRIPTION OF THE INVENTION

The present invention is based upon the discovery that agents that increase hepatic levels of nitric oxide may be used to prevent liver damage in patients that have overingested acetaminophen. In referring to toxic effects caused by acetaminophen, it will be understood the risk of liver damage may be the result of a patient ingesting any drug product containing this drug and that the acetaminophen may be in any form found in such a drug product.

In a first aspect, the invention is directed to a method of treating a patient at risk for liver damage due to the excessive ingestion of acetaminophen by the systemic or local administration of a therapeutically effective amount of a compound that increases hepatic levels of nitric oxide. A patient at risk for liver damage due to the excessive ingestion of acetaminophen is any patient that is judged by a physician, nurse, other medical practitioner or public health worker to be at risk, taking into consideration the amount of drug taken as well as any medical factors that may be specific for that patient. Typically, treated patients will be individuals ingesting more than at least 2.0 grams of the drug within a 24 hour period. However, clinical considerations may sometimes result in a medical practitioner judging others to be at risk as well. For example, if a patient already has liver disease or is taking medications that may make damage to the liver more likely, a physician may treat the patient as described herein as a precaution even if they have taken a lower amount of acetaminophen. Other factors that a practitioner may consider are age, medical history, alcohol use etc. For the purposes of the invention, any individual that has ingested more than 4.0 g of acetaminophen within a 24 hour period or an average of more than 2.0 g per day for 5 or more consecutive days is considered at risk of liver damage and is part of the patients encompassed by the present methods regardless of whether they have been diagnosed by a medical practitioner as being at risk or not.

At the time of treatment, the patient may, or may not, be exhibiting one or more signs or symptoms associated with liver toxicity, liver damage or hepatic necrosis. For the purposes of the present invention symptoms associated with liver toxicity include vomiting, nausea, pallor or sweating within about 24 hours of ingestion; signs of liver damage include right-upper-quadrant pain or abnormalities in one or more biomarkers of liver function between about 24 and 72 hours after ingestion; and signs associated with hepatic necrosis include hypoglycemia, kidney failure, hepatic encephalopathy, cerebral edema, sepsis, and multiple organ failure between about 3 to 5 days after ingestion. In all cases, a patient should be treated as soon as it is determined that they are at risk and no later than one week after the ingestion of the acetaminophen causing the patient to be at risk. Preferably, treatment is initiated within 72 hours of ingestion.

The compounds administered to a patient may be any therapeutically acceptable NO donor or compound that otherwise acts to increase patient NO levels. Many appropriate compounds are known in the literature and are reviewed herein. The compounds include L-arginine, citrulline, ornithine, glutamine, lysine, S-nitrosothiols, and, most preferably, inhibitors of S-nitrosoglutathione reductase.

Dosages will be determined by attending medical practitioners and will depend upon clinical factors such as the age and medical history of the patient as well as the amount of acetaminophen taken and the compound being administered. Typically, a patient will be given 10 μg to 100 mg of a compound per kg body weight per day for at least one day. This may be repeated for as many days as deemed prudent to prevent damage or until symptoms of liver toxicity have passed and/or liver function tests have returned to normal.

Treatment with the compounds described herein may also be combined with other treatments appropriate for a patient. In this regard, therapies aimed at increasing hepatic nitric oxide levels may be combined with the administration of N-acetylcysteine, a standard therapy for acetaminophen poisoning. It is believed that combining these therapies may produce a protective effect that is more beneficial than when either therapy is used alone

Preferably, the compounds described herein will be administered in unit dose form to patients as part of a pharmaceutical composition. Preferred routes of administration are infusion, intravenous injection or orally as a tablet or capsule.

### DETAILED DESCRIPTION OF THE INVENTION

A. Compounds

Compounds that may be used in this invention include nitric oxide donors or compounds that act metabolically to enhance the activity of nitric oxide synthases, increase nitrosothiol levels, inhibit the conversion of nitric oxide to S-nitrosoglutathione or that block the reduction of S-nitrosglutathione by S-nitrosglutathione reductase.

Nitric oxide donor compounds are described in U.S. Pat. Nos. 4,954,526; 5,155,137; 5,212,204; 5,250,550; 5,525,357; 5,366,997; 5,380,758; 5,405,919; 5,650,447; 5,859,053; 5,703,073; 5,039,705; 5,910,316; 6,297,260; 6,232,336; 7,1968,575; 8,057,464 and in WO 94/03421; WO 94/04484; WO 94/12463; WO 95/09831; WO 95/19952; WO 95/30641; WO 97/27749; WO 98/19672; WO 98/21193; WO 00/51988; WO 00/61604; WO 00/72838; WO 01/00563; WO 01/04082; WO 01/10814; WO 01/12584; WO 01/45703; WO 00/61541; WO 00/61537; WO 02/11707; and WO 02/30866, all of which are hereby incorporated by reference in their entirety. The compounds include S-nitrosothiols, nitrites, nitrates, S-nitrothiols, 2-hydroxy-2-nitrosohydrazines, (NONOates), N-nitrosoamines, N-hydroxyl nitrosamines, nitrosimines, diazetine dioxides, oxatriazole 5-imines, oximes, hydroxylamines, N-hydroxyguanidines, hydroxyureas, benzofuroxanes, furoxans etc.

The invention is also directed to compounds that stimulate the production of endogenous NO levels, e.g., L-arginine, citrulline, ornithine, glutamine, lysine and to agents that block enzymes that lead to the breakdown of compounds that are substrates for the production of NO. Of these, the most preferred are the inhibitors of S-nitrosoglutathione reductase that are described by Stamler, et al. in U.S. Pat. Nos. 7,179,791; 7,615,535; and 2010/0015121; and especially the pyrrole inhibitors disclosed by Wasley, et al. in US 2011/0144110 and US 2011/0144180 or related international applications WO 2010/019903 and WO 2010/019910, all of which are hereby incorporated by reference in their entirety. The most preferred S-nitrosoglutathione reductase inhibitor is N6547, owned by N30 Pharmaceuticals.

Inhibitors of S-nitrosoglutathione reductase (glutathione-dependent formaldehyde dehydrogenase) described by Stamler, et al. include: nicotinamide riboside; and ribonucleoside analogs such as: 6-aminonicotinamide; 5-β-D-ribofuranosylnicotin-amide; 6-β-D-ribofuranosylisonicotinamide; 2-β-D-ribofuranosylpicolinamide; thiophenfurin; furanfurin; tiazofurin; selenazofurin; selenophenfurin. Stamler also discloses the inhibitors benzamide ribosome; ribavirin; mizoribine; 5-ethynyl-1-β-D-ribofuranosylimidazole-4-carboxamide; 6-amino-NAD; 5-β-D-ribofuranosylnicotinamide adenine dinucleotide; 6-β-D-ribofuranosylpicolinamide adenine dinucleotide; benzamide adenine dinucleotide; β-CH₂-TAD; β-CF₂-TAD; 3′F-TAD; 2′Fara-TAD; 2′Fara-β-CH₂-TAD; 2′Fara-β-CF₂-TAD; β-CH2-BAD; mycophenolic acid; mycophenolate mofetil; 6-thioanologs of natural purine bases, such as 6-mercaptopurine and 6-thioguanine; glutathione derivatives including D-glutathione and S-alkyl glutathione containing from 1 to 6 carbon atoms in the S-alkyl group.

Inhibitors of S-nitrosoglutathione reductase disclosed in WO 2010/019903 include compounds of formula I:

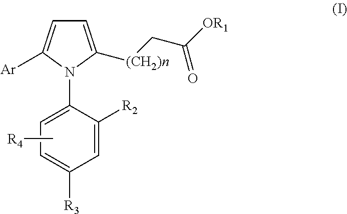

wherein
Ar is selected from the group consisting of aryl, substituted aryl, heteroaryl and substituted heteroaryl;
R₁ is selected from the group consisting of hydrogen, C₁-C₆ alkyl, C₃-C₆ cycloalkyl, aryl, substituted aryl, heteroaryl, and substituted heteroaryl;
R₂ is selected from the group consisting of halogen, C₁-C₆ alkyl, C₃-C₆ cycloalkyl, cyano, nitro, CF₃, carbamoyl, C₁-C₆ alkylcarbamoyl, amino, C₁-C₆ alkylamino, C₁-C₆ dialkylamino, C₁-C₆ alkoxyl, and C3-C6 cycloalkoxyl;
R₃ is selected from the group consisting of hydroxyl, carbamoyl, C₁-C₆ alkylcarbamoyl, sulfamoyl, C₁-C₆ alkylsulfamoyl, C₃-C₆ cycloalkyl, cyano, nitro, carboxyl, amino, aminomethyl, acetamido, acetamido-2-methyl, 2-methoxy-acetamido, propionamido, tetrahydrofuran-2-ylmethoxy, CF₃, carboxyl, ureido, sulfamoylamino, C₁-C₆ alkylsulfonamido, 2-amino-2-oxoethyl, C₁-C₆ alkylamino, C₁-C₆ dialkylamino, arylamino, heteroarylamino, C₁-C₆ alkoxyl, C₃-C₆ cycloalkoxyl, 1H-imidazol-2-yl, oxazol-2-yl, thiazol-2-yl, methylcarbamoyl, dimethycarbamoyl, methoxyethylcarbamoyl, hydroxyethylcarbamoyl, (dimethylamino)ethylcarbamoyl, pyridin-3-ylcarbamoyl, pyridin-4-yl-carbamoyl, 6-methoxypyridin-3-ylcarbamoyl, pyridazin-4-ylcarbamoyl, and pyrimidin-5-ylcarbamoyl;
R₄ is selected from the group consisting of hydrogen, hydroxyl, halogen, C₁-C₆ alkyl, C₃-C₆ cycloalkyl, cyano, nitro, carbamoyl, C₁-C₆ alkylcarbamoyl, sulfamoyl, C₁-C₆ alkyl sulfamoyl, amino, C1-C₆ alkylamino, C₁-C₆ dialkylamino, C₁-C₆ alkoxyl, and C₃-C₆ cycloalkoxyl;
n is 0-3;
wherein Ar excludes substituted or unsubstituted indazol-3-yl and substituted or unsubstituted 1H-pyrazolo[3,4-b]pyridyl;
with the following provisos:
proviso Ia: when R₃ is carboxyl, methylsulfonamido, or sulfamoyl, then Ar cannot be phenyl, substituted or unsubstituted phenyl-(C₁-C₃)-alkyl, or phenyl-(C₂-C₆)-alkenyl; and
proviso Ib: when R₂ and R₃ are both methoxy, then Ar cannot be phenyl, 4-methyl-phenyl, 4-methoxy-phenyl, 4-halo-phenyl, or thiophen-yl.

Specific compounds within the scope of formula I of WO 2010/019903 include: 3-(5-(4-(1H-imidazol-1-yl)phenyl)-1-(4-carbamoyl-2-methylphenyl)-1H-pyrrol-2-yl)propanoic acid; 3-(5-(5-(1H-imidazol-1-yl)thiophen-2-yl)-1-(4-carbamoyl-2-methylphenyl)-1H-pyrrol-2-yl)propanoic acid; 3-(1-(4-carbamoyl-2-methylphenyl)-5-(4-(2-methyl-1H-imidazol-yl)phenyl)-1H-pyrrol-2-yl)propanoic acid; 3-(1-(4-carbamoyl-2-methylphenyl)-5-(thiazol-5-yl)-1H-pyrrol-2-yl)propanoic acid; 3-(1-(4-carbamoyl-2-methylphenyl)-5-(4-hydroxyphenyl)-1H-pyrrol-2-yl)propanoic acid; 3-(5-(5-bromothiophen-2-yl)-1-(4-carbamoyl-2-methylphenyl)-1H-pyrrol-2-yl)propanoic acid; 3-(1-(4-carbamoyl-2,6-dimethylphenyl)-5-(4-methoxyphenyl)-1H-pyrrol-2-yl)propanoic acid; 3-(5-(4-(1H-imidazol-1-yl)phenyl)-1-(4-hydroxy-2-methylphenyl)-1H-pyrrol-2-yl)propanoic acid; 3-(1-(4-carbamoyl-2-methylphenyl)-5-(4-methoxyphenyl)-1H-pyrrol-2-yl)propanoic acid; 3-(1-(4-carbamoyl-2-chlorophenyl)-5-(4-methoxyphenyl)-1H-pyrrol-2-yl)propanoic acid; 3-(5-(4-aminophenyl)-1-(4-carbamoyl-2-methylphenyl)-1H-pyrrol-2-yl)propanoic acid; 3-(5-(4-bromophenyl)-1-(4-carbamoyl-2-methylphenyl)-1H-pyrrol-2-yl)propanoic acid; 3-(1-(4-carbamoyl-2-methylphenyl)-5-(4-(furan-3-yl)phenyl)-1H-pyrrol-2-yl)propanoic acid; 3-(5-(4-methoxyphenyl)-1-(2-methyl-4-sulfamoylphenyl)-1H-pyrrol-2-yl)propanoic acid; 3-(1-(4-carbamoyl-2-methylphenyl)-5-phenyl-1H-pyrrol-2-yl)propanoic acid; 3-(1-(4-carbamoyl-2-methylphenyl)-5-(3-chloro-4-methoxyphenyl)-1H -pyrrol-2-yl)propanoic acid; 3-(1-(4-carbamoyl-2-methylphenyl)-5-(3-fluoro-4-methoxyphenyl)-1H-pyrrol-2-yl)propanoic acid; 3-(1-(4-carbamoyl-2-methylphenyl)-5-(4-cyanophenyl)-1H-pyrrol-2-yl)propanoic acid; 3-(1-(4-carbamoyl-2-methylphenyl)-5-(3-chloro-4-hydroxyphenyl)-1H-pyrrol-2-yl)propanoic acid; 3-(1-(4-carbamoyl-2-ethylphenyl)-5-(4-methoxyphenyl)-1H-pyrrol-2-yl)propanoic acid; 3-(1-(4-carbamoyl-2-methylphenyl)-5-(4-methoxy-3-methylphenyl)-1H-pyrrol-2-yl)propanoic acid; 3-(1-(4-carbamoyl-2-methylphenyl)-5-(4-iodophenyl)-1H-pyrrol-2-yl)propanoic acid; 3-(1-(4-carbamoyl-2-methylphenyl)-5-(3-methoxyphenyl)-1H-pyrrol-2-yl)propanoic acid; 3-(1-(4-carbamoyl-2-methylphenyl)-5-(6-methoxypyridin-3-yl)-1H-pyrrol-2-yl)propanoic acid; 3-(5-(benzo[d]thiazol-6-yl)-1-(4-carbamoyl-2-methylphenyl)-1H-pyrrol-2-yl)propanoic acid; 3-(1-(4-carbamoyl-2-methylphenyl)-5-(4-(methylthio) phenyl)-1H-pyrrol-2-yl)propanoic acid; 3-(1-(4-carbamoyl-2-methylphenyl)-5-(4-nitrophenyl)-1H-pyrrol-2-yl)propanoic acid; 3-(5-(4-(1H-imidazol-1-yl)phenyl)-1-(4-amino-2-methylphenyl)-1H-pyrrol-2-yl)propanoic acid; 3-(5-(4-(4H-1,2,4-triazol-4-yl)phenyl)-1-(4-carbamoyl-2-methyIphenyl)-1H-pyrrol-2-yl)propanoic acid; 3-(5-(4-(2H-tetrazol-5-yl)phenyl)-1-(4-carbamoyl-2-methylphenyl)-1H-pyrrol-2-yl)propanoic acid; 3-(1-(4-methoxy-2-methylphenyl)-5-(4-methoxyphenyl)-1H-pyrrol-2-yl)propanoic acid; 3-(5-(benzo[d] [1,3]dioxol-5-yl)-1-(4-carbamoyl-2-methylphenyl)-1H-pyrrol-2-yl)propanoic acid; 3-(5-(4-bromophenyl)-1-(4-hydroxy-2-methylphenyl)-1H-pyrrol-2-yl)propanoic acid; 3-(5-(6-(1H-imidazol-1-yl)pyridin-3-yl)-1-(4-carbamoyl-2-methylphenyl)-1H-pyrrol-2-yl)propanoic acid; 3-(1-(4-carbamoyl-2-methylphenyl)-5-(5-carbamoylthiophen-2-yl)-1H-pyrrol-2-yl)propanoic acid; 3-(5-(4-(1H-pyrazol-4-yl)phenyl)-1-(4-carbamoyl-2-methylphenyl)-1H-pyrrol-2-yl)propanoic acid; 3-(1-(4-carbamoyl-2-methylphenyl)-5-(4-(trifluoromethoxy)phenyl)-1H-pyrrol-2-yl)propanoic acid; 3-(1-(4-carbamoyl-2-methylphenyl)-5-(4-carbamoylphenyl)-1H-pyrrol-2-yl)propanoic acid; 3-(1-(4-carbamoyl-2-methylphenyl)-5-(4-(trifluoromethyl)phenyl)-1H-pyrrol-2-yl)propanoic acid; 3-(5-(4-(1H-1,2,4-triazol-1-yl)phenyl)-1-(4-carbamoyl-2-methylphenyl)-1H-pyrrol-2-yl)propanoic acid; 3-(5-(4-(1H-pyrazol-1yl)phenyl)-1-(4-carbamoyl-2-methylphenyl)-1H-pyrrol-2-yl)propanoic acid; 3-(5-(4-(2-amino-2-oxoethoxy)phenyl)-1-(4-carbamoyl-2-methylphenyl)-1H-pyrrol-2-yl)propanoic acid; 3-(1-(4-carbamoyl-2-methylphenyl)-5-(4-(1-methyl-1H -pyrazol-4-yl)phenyl)-1H -pyrrol-2-yl)propanoic acid; 3-(5-(4-(1H-tetrazol-1-yl)phenyl)-1-(4-carbamoyl-2-methylphenyl)-1H-pyrrol-2-yl)propanoic acid; 3-(1-(4-carbamoyl-2-methylphenyl)-5-(4-(4-methyl-1H-imidazol-1yl)phenyl)-1H-pyrrol-2-yl)propanoic acid; 3-(1-(4-carbamoyl-2-methylphenyl)-5-(4-ureidophenyl)-1H-pyrrol-2-yl)propanoic acid; 4-(2-(benzo[d] [1,3]dioxol-5-yl)-5-(2-carboxyethyl)-1H-pyrrol-1-yl)-3-methylbenzoic acid; 3-(1-(4-amino-2-methylphenyl)-5-(4-bromophenyl)-1H-pyrrol-2-yl)propanoic acid; 3-(1-(4-carbamoyl-2-methylphenyl)-5-(4-morpholinophenyl)-1H-pyrrol-2-yl)propanoic acid; 4-(2-(4-(2H-tetrazol-5-yl)phenyl)-5-(2-carboxyethyl)-1H-pyrrol-1-yl)-3-methylbenzoic acid; 3-(1-(4-carbamoyl-2-methylphenyl)-5-(4-(pyridin-4-yl)phenyl)-1H-pyrrol-2-yl)propanoic acid; 3-(5-(4-(1H-pyrrol-1-yl)phenyl)-1-(4-carbamoyl-2-methylphenyl)-1H-pyrrol-2-yl)propanoic acid; 3-(1-(4-hydroxy-2,5-dimethylphenyl)-5-(4-methoxyphenyl)-1H-pyrrol-2-yl)propanoic acid; 3-(1-(4-carbamoyl-2-methylphenyl)-5-(4-(methylsulfonamido)phenyl)-1H-pyrrol-2-yl)propanoic acid; 3-(5-(4-(1H-1,2,3-triazol-1-yl)phenyl)-1-(4-carbamoyl-2-methylphenyl)-1H-pyrrol-2-yl)propanoic acid; 3-(1-(4-carbamoyl-2-methylphenyl)-5-(naphthalen-2-yl)-1H-pyrrol-2-yl)propanoic acid; 3-(1-(4-carbamoyl-2-methylphenyl)-5-(3′-carbamoylbiphenyl-4-yl)-1H-pyrrol-2-yl)propanoic acid; 3-(1-(4-carbamoyl-2-methylphenyl)-5-(4-(6-oxo-1,6-dihydropyridin-3-yl)phenyl)-1H-pyrrol-2-yl)propanoic acid; 3-(1-(4-carbamoyl-2-methylphenyl)-5-(4-(3,5-dimethylisoxazol-4-yl)phenyl)-1H-pyrrol-2-yl)propanoic acid;3-(1-(4-carbamoyl-2-methylphenyl)-5-(4-(pyridin-3-yl)phenyl)-1H -pyrrol-2-yl)propanoic acid;3-(1-(4-carbamoyl-2-methylphenyl)-5-(4-(carboxymethoxy)phenyl)-1H-pyrrol-2-yl)propanoic acid;3-(1-(4-carbamoyl-2-methylphenyl)-5-(4-(2-hydroxyacetamido)phenyl)-1H-pyrrol-2-yl)propanoic acid; 3-(1-(4-carbamoyl-2-methylphenyl)-5-(4-(2-methoxyacetamido)-phenyl)-1H-pyrrol-2-yl)propanoic acid; 3-(1-(4-carbamoyl-2-methylphenyl)-5-(4-sulfamoylphenyl)-1H-pyrrol-2-yl)propanoic acid; 3-(1-(4-carbamoyl-2-methylphenyl)-5-(4-(piperazin-1-yl)phenyl)-1H-pyrrol-2-yl)propanoic acid; 3-(1-(4-carbamoyl-2-methylphenyl)-5-(4-(2-oxoimidazolidin-1yl)phenyl)-1H-pyrrol-2-yl)propanoic acid; 3-(1-(4-carbamoyl-2-methylphenyl)-5-(4-ethoxyphenyl)-1H-pyrrol-2-yl)propanoic acid; 3-(1-(4-carbamoyl-2-methylphenyl)-5-(4-(methylsulfinyl)phenyl)-1H -pyrrol-2-yl)propanoic acid; 3-(1-(4-carbamoyl-2-methylphenyl)-5-(4-(2-ethyl-1H-imidazol-1-yl)phenyl)-1H-pyrrol-2-yl)propanoic acid; 3-(5-(4-(1H-imidazol-1-yl)phenyl)-1-(4-(aminomethyl)-2-methylphenyl)-1H-pyrrol-2-yl)propanoic acid; 3-(1-(4-(1H-imidazol-2-yl)-2-methylphenyl)-5-(4-methoxyphenyl)-1H-pyrrol-2-yl)propanoic acid; 3-(1-(4-carbamoyl-2-methylphenyl)-5-(4-(furan-2-yl)phenyl)-1H-pyrrol-2-yl)propanoic acid; 3-(1-(4-(dimethylcarbamoyl)-2-methylphenyl)-5-(4-methoxyphenyl)-1H-pyrrol-2-yl)propanoic acid; 3-(5-(4-methoxyphenyl)-1-(2-methyl-4-(methylcarbamoyl)phenyl)-1H -pyrrol-2-yl)propanoic acid; 3-(1-(4-(2-methoxyethylcarbamoyl)-2-methylphenyl)-5-(4-methoxyphenyl)-1H-pyrrol-2-yl)propanoic acid; 3-(1-(4-(2-hydroxyethylcarbamoyl)-2-methylphenyl)-5-(4-methoxyphenyl)-1H-pyrrol-2-yl)propanoic acid; 3-(5-(4-methoxyphenyl)-1-(2-methyl-4-(oxazol-2-yl)phenyl)-1H-pyrrol-2-yl)propanoic acid; 3-(1-(4-carbamoyl-2-methylphenyl)-5-(4-(methylsulfonyl)phenyl)-1H-pyrrol-2-yl)propanoic acid; 3-(1-(4-hydroxy-2-methylphenyl)-5-(4-methoxyphenyl)-1H-pyrrol-2-yl)propanoic acid; 3-(1-(2-chloro-4-hydroxyphenyl)-5-(4-methoxyphenyl)-1H -pyrrol-2-yl)propanoic acid; 3-(1-(4-hydroxy-2,3-dimethylphenyl)-5-(4-methoxyphenyl)-1H -pyrrol-2-yl)propanoic acid; 3-(1-(4-carbamoyl-2-methylphenyl)-5-(4-(pyrrolidin-1-yl)phenyl)-1H-pyrrol-2-yl)propanoic acid; 3-(1-(4-carbamoyl-2-methylphenyl)-5-(4-(piperidin-1-yl)phenyl)-1H-pyrrol-2-yl)propanoic acid; 3-(1-(4-carbamoyl-2-methylphenyl)-5-(4-(1-methyl-1H-pyrazol-5-yl)phenyl)-1H-pyrrol-2-yl)propanoic acid; ethyl 3-(5-(4-(1H-imidazol-1-yl)phenyl)-1-(4-carbamoyl-2-methylphenyl)-1H-pyrrol-2-y1)propanoate; ethyl 3-(1-(4-carbamoyl-2-methylphenyl)-5-(4-(2-methyl-1H-imidazol-1-y1)phenyl)-1H-pyrrol-2-yl)propanoate;ethyl 3-(1-(4-carbamoyl-2-methylphenyl)-5-(5-(2-methyl-1H-imidazol-1-yl)thiophen-2-yl)-1H-pyrrol-2-yl)propanoate; ethyl 3-(1-(4-carbamoyl-2-methylphenyl)-5-(4-(furan-3-yl)phenyl)-1H-pyrrol-2-yl)propanoate; ethyl 3-(1-(4-carbamoyl-2-methylphenyl)-5-(4-cyanophenyl)-1H-pyrrol-2-yl)propanoate; ethyl 3-(1-(4-carbamoyl-2-methylphenyl)-5-(4-(furan-2-yl)phenyl)-1H-pyrrol-2-yl)propanoate; 3-(1-(4-carbamoyl-2-methylphenyl)-5-(2-methoxypyrimidin-5-yl)-1H-pyrrol-2-yl)propanoic acid; 3-(5-(4-(1H-imidazol-1-yl)phenyl)-1-(2-methyl-4-(methylsulfonamido)phenyl)-1H-pyrrol-2-yl)propanoic acid; 3-(5-(4-(1H-imidazol-1-yl)phenyl)-1-(4-acetamido-2-methylphenyl)-1H-pyrrol-2-yl)propanoic acid; 3-(1-(4-hydroxy-2-(trifluoromethyl)phenyl)-5-(4-methoxyphenyl)-1H-pyrrol-2-yl)propanoic acid; 3-(5-(4-methoxyphenyl)-1-(4-((tetrahydrofuran-2-yl)methoxy)-2-(trifluoromethyl)phenyl)-1H -pyrrol-2-yl)propanoic acid; 3-(1-(4-carbamoyl-2-(trifluoromethyl)phenyl)-5-(4-methoxyphenyl)-1H-pyrrol-2-yl)propanoic acid; 3-(5-(4-(1H-imidazol-1-yl)phenyl)-1-(2-methyl-4-propionamidophenyl)-1H-pyrrol-2-yl)propanoic acid; 3-(5-(4-(1H-imidazol-1-yl)phenyl)-1-(4-(2-methoxyacetamido)-2-methylphenyl)-1H-pyrrol-2-yl)propanoic acid; 3-(5-(4-amino-3-chlorophenyl)-1-(4-carbamoyl-2-methylphenyl)-1H-pyrrol-2-yl)propanoic acid; 3-(1-(4-carbamoyl-2-methylphenyl)-5-(3,4-difluorophenyl)-1H-pyrrol-2-yl)propanoic acid; 3-(1-(4-carbamoyl-2-methylphenyl)-5-(2,4-difluorophenyl)-1H-pyrrol-2-yl)propanoic acid; 3-(1-(4-carbamoyl-2-methylphenyl)-5-(4-chlorophenyl)-1H-pyrrol-2-yl)propanoic acid; 3-(5-(4-bromothiophen-2-yl)-1-(4-carbamoyl-2-methylphenyl)-1H-pyrrol-2-yl)propanoic acid; 3-(5-(biphenyl-4-yl)-1-(4-carbamoyl-2-methylphenyl)-1H-pyrrol-2-yl)propanoic acid; 3-(1-(4-carbamoyl-2-fluorophenyl)-5-(4-methoxyphenyl)-1H-pyrrol-2-yl)propanoic acid; 3-(5-(2-(1H-imidazol-1-yl)pyrimidin-5-yl)-1-(4-carbamoyl-2-methylphenyl)-1H-pyrrol-2-yl)propanoic acid; 3-(1-(4-carbamoyl-2-methylphenyl)-5-(4-fluoro-3-methoxyphenyl)-1H -pyrrol-2-yl)propanoic acid; 3-(1-(4-carbamoyl-2-methylphenyl)-5-(3-cyano-4-fluorophenyl)-1H-pyrrol-2-yl)propanoic acid; 3-(1-(4-carbamoyl-2-methylphenyl)-5-(4-carbamoyl-3-fluorophenyl)-1H-pyrrol-2-yl)propanoic acid; 3-(1-(4-carbamoyl-2-methylphenyl)-5-(4-methoxy-2-methylphenyl)-1H-pyrrol-2-yl)propanoic acid; 3-(5-(4-methoxyphenyl)-1-(2-methyl-4-(thiazol-2-yl)phenyl)-1H-pyrrol-2-yl)propanoic acid; 3-(1-(4-carbamoyl-2-methylphenyl)-5-(4-chloro-2-fluorophenyl)-1H-pyrrol-2-yl)propanoic acid; 3-(1-(4-carbamoyl-2-methylphenyl)-5-(4-fluorophenyl)-1H-pyrrol-2-yl)propanoic acid; 3-(1-(4-carbamoyl-2-methylphenyl)-5-(4-fluoro-2-methylphenyl)-1H-pyrrol-2-yl)propanoic acid; 3-(1-(4-carbamoyl-2-methylphenyl)-5-(4-chloro-2-(trifluoromethyl)phenyl)-1H-pyrrol-2-yl)propanoic acid; 3-(1-(4-carbamoyl-2-methylphenyl)-5-(4-chloro-2-methoxyphenyl)-1H-pyrrol-2-yl)propanoic acid; 3-(1-(4-carbamoyl-2-methylphenyl)-5-(4-cyano-3-fluorophenyl)-1H-pyrrol-2-yl)propanoic acid; 3-(1-(4-carbamoyl-2-methylphenyl)-5-(2-chloro-4-methoxyphenyl)-1H -pyrrol-2-yl)propanoic acid; 3-(1-(4-(2-(dimethylamino)ethylcarbamoyl)-2-methylphenyl)-5-(4-methoxyphenyl)-1H-pyrrol-2-yl)propanoic acid; 3-(5-(4-(1H-imidazol1yl)thiophen-2-yl)-1-(4-carbamoyl-2-methylphenyl)-1H-pyrrol-2-yl)propanoic acid; 3-(1-(4-carbamoyl-2-methylphenyl)-5-(3,5-dimethyl-4-nitrophenyl)-1H-pyrrol-2-yl)propanoic acid; 3-(5-(4-amino-3,5-dimethylphenyl)-1-(4-carbamoyl-2-methylphenyl)-1H-pyrrol-2-yl)propanoic acid;3-(5-(2-(1H-imidazol-1-yl)thiazol-4-yl)-1-(4-carbamoyl-2-methylphenyl)-1H-pyrrol-2-yl)propanoic acid; 3-(1-(4-carbamoyl-2-methylphenyl)-5-(2-ethoxy-4-fluorophenyl)-1H-pyrrol-2-yl)propanoic acid; 3-(1-(4-carbamoyl-2-methylphenyl)-5-(4-methoxy-2-(trifluoromethyl)phenyl)-1H-pyrrol-2-yl)propanoic acid; 3-(1-(4-carbamoyl-2-methylphenyl)-5-(4-fluoro-2-methoxyphenyl)-1H-pyrrol-2-yl)propanoic acid; 3-(5-(4-bromophenyl)-1-(2-methyl-4-(pyridin-3-ylcarbamoyl)phenyl)-1H-pyrrol-2-yl)propanoic acid; 3-(5-(4-bromophenyl)-1-(4-(6-methoxypyridin-3-ylcarbamoyl)-2-methylphenyl)-1H -pyrrol-2-yl)propanoic acid; 3-(5-(4-methoxyphenyl)-1-(4-(6-methoxypyridin-3-ylcarbamoyl)-2-methylphenyl)-1H-pyrrol-2-yl)propanoic acid; 3-(1-(4-carbamoyl-2-methylphenyl)-5-(4-(thiazol-5-yl)phenyl)-1H-pyrrol-2-yl)propanoic acid; 3-(1-(4-carbamoyl-2-methylphenyl)-5-(4-chloro-3-fluorophenyl)-1H-pyrrol-2-yl)propanoic acid; 3-(1-(4-carbamoyl-2-methylphenyl)-5-(4-(hydroxycarbamoyl)phenyl)-1H-pyrrol-2-yl)propanoic acid; 3-(5-(4-bromophenyl)-1-(2-methyl-4-(pyridin-4-ylcarbamoyl)phenyl)-1H-pyrrol-2-yl)propanoic acid; 3-(5-(4-methoxyphenyl)-1-(2-methyl-4-(pyridazin-4-ylcarbamoyl)phenyl)-1H-pyrrol-2-yl)propanoic acid; 3-(5-(4-methoxyphenyl)-1-(2-methyl-4-(pyridin-3-ylcarbamoyl)phenyl)-1H -pyrrol-2-yl)propanoic acid; 3-(1-(4-carbamoyl-2-methylphenyl)-5-(5-(2-methyl-1H-imidazol-1-yl)thiophen-2-yl)-1H-pyrrol-2-yl)propanoic acid;3-(1-(4-carbamoyl-2-methylphenyl)-5-(3-fluoro-4-(1H-imidazol-1-yl)phenyl)-1H-pyrrol-2-yl)propanoic acid; 3-(5-(4-methoxyphenyl)-1-(2-methyl-4-(pyrimidin-5-ylcarbamoyl)phenyl)-1H-pyrrol-2-yl)propanoic acid; 3-(5-(4-(2-methyl-1H-imidazol-1-yl)phenyl)-1-(2-methyl-4-(methylsulfonamido)phenyl)-1H-pyrrol-2-yl)propanoic acid; 3-(1-(4-carbamoyl-2-methylphenyl)-5-(3-fluoro-4-(2-methyl-1H-imidazol-1-yl)phenyl)-1H-pyrrol-2-yl)propanoic acid; 3-(1-(4-carbamoyl-2-methylphenyl)-5-(4-chloro-2-ethoxyphenyl)-1H-pyrrol-2-yl)propanoic acid; 3-(5-(5-bromo-2-methoxyphenyl)-1-(4-carbamoyl-2-methylphenyl)-1H-pyrrol-2-yl)propanoic acid; 3-(1-(4-carbamoyl-2-methylphenyl)-5-(4-(2-cyclopropyl-1H-imidazol-1yl) phenyl)-1H-pyrrol-2-yl)propanoic acid; 3-(1-(4-carbamoyl-2-methylphenyl)-5-(4-(2-methyl-1H-imidazol-1-yl)thiophen-2-yl)-1H-pyrrol-2-yl)propanoic acid; 3-(5-(4-bromo-2-methoxyphenyl)-1-(4-carbamoyl-2-methylphenyl)-1H-pyrrol-2-yl)propanoic acid; 3-(1-(4-carbamoyl-2-methylphenyl)-5-(2-methoxy-4-(2-methyl-1H -imidazol-l-yl)phenyl)-1H -pyrrol-2-yl)propanoic acid; 3-(1-(4-acetamido-2-methylphenyl)-5-(4-bromophenyl)-1H-pyrrol-2-yl)propanoic acid; 3-(5-(4-bromophenyl)-1-(4-(2-methoxyacetamido)-2-methylphenyl)-1H-pyrrol-2-yl)propanoic acid; 3-(1-(4-carbamoyl-2-methylphenyl)-5-(4-chloro-2-hydroxyphenyl)-1H-pyrrol-2-yl)propanoic acid; 3-(5-(5-bromothiophen-3-yl)-1-(4-carbamoyl-2-methylphenyl)-1H-pyrrol-2-yl)propanoic acid; 3-(1-(4-carbamoyl-2-methylphenyl)-5-(4-hydroxy-3-methylphenyl)-1H-pyrrol-2-yl)propanoic acid; 3-(1-(4-carbamoyl-2-methylphenyl)-5-(2-carbamoyl-4-chlorophenyl)-1H-pyrrol-2-yl)propanoic acid; 3-(5-(2-acetamido-4-chlorophenyl)-1-(4-carbamoyl-2-methylphenyl)-1H-pyrrol-2-yl)propanoic acid; 3-(1-(4-carbamoyl-2-methylphenyl)-5-(2-methoxyphenyl)-1H-pyrrol-2-yl)propanoic acid; 4-(1-(4-carbamoyl-2-methylphenyl)-5-(2-carboxyethyl)-1H-pyrrol-2-yl)benzoic acid; 3-(1-(4-carbamoyl-2-methylphenyl)-5-(2,4-dimethoxyphenyl)-1H-pyrrol-2-yl)propanoic acid; 3-(1-(4-carbamoyl-2-methylphenyl)-5-(4-chloro-2-propoxyphenyl)-1H-pyrrol-2-yl)propanoic acid; 3-(1-(4-carbamoyl-2-methylphenyl)-5-(4-chloro-2-(2-methoxyacetamido)phenyl)-1H-pyrrol-2-yl)propanoic acid; 3-(1-(4-carbamoyl-2-methylphenyl)-5-(4-chloro-2-(methylsulfonamido)phenyl)-1H-pyrrol-2-yl)propanoic acid; 3-(1-(4-carbamoyl-2-methylphenyl)-5-(4-chloro-2-(methylamino)phenyl)-1H-pyrrol-2-yl)propanoic acid; 3-(1-(4-carbamoyl-2-methylphenyl)-5-(4-chloro-2-(methoxymethyl)phenyl)-1H-pyrrol-2-yl)propanoic acid; 3-(1-(4-carbamoyl-2-methylphenyl)-5-(4-hydroxy-2-methoxyphenyl)-1H-pyrrol-2-yl)propanoic acid; 3-(1-(4-carbamoyl-2-methylphenyl)-5-(4-chloro-2-(dimethylamino)phenyl)-1H-pyrrol-2-yl)propanoic acid; 3-(1-(4-carbamoyl-2-methylphenyl)-5-(4-(2-(hydroxymethyl)-1H-imidazol-1-yl)phenyl)-1H-pyrrol-2-yl)propanoic acid; 3-(1-(4-carbamoyl-2-methylphenyl)-5-(4-(2-oxooxazolidin-3-yl)phenyl)-1H-pyrrol-2-yl)propanoic acid; 3-(5-(4-(1H-imidazol-1-yl)-2-methoxyphenyl)-1-(4-carbamoyl-2-methylphenyl)-1H-pyrrol-2-yl)propanoic acid; 3-(1-(4-carbamoyl-2-methylphenyl)-5-(5-(2-methyl-1H-imidazol-1-yl)thiophen-3-yl)-1H-pyrrol-2-yl)propanoic acid; 3-(1-(4-carbamoyl-2-methylphenyl)-5-(5-chlorothiophen-2-yl)-1H-pyrrol-2-yl)propanoic acid; 3-(5-(5-bromothiazol-2-yl)-1-(4-carbamoyl-2-methylphenyl)-1H-pyrrol-2-yl)propanoic acid; 3-(5-(4-(2-methyl-1H-imidazol-1-yl)thiophen-2-yl)-1-(2-methyl-4-(methylsulfonamido)phenyl)-1H-pyrrol-2-yl)propanoic acid; 3-(5-(5-(2-methyl-1H-imidazol-1-yl)thiophen-2-yl)-1-(2-methyl-4-(methylsulfonamido)phenyl)-1H-pyrrol-2-yl)propanoic acid; 3-(1-(4-acetamido-2-methylphenyl)-5-(4-chloro-2-methoxyphenyl)-1H-pyrrol-2-yl)propanoic acid; 3-(5-(4-bromothiazol-2-yl)-1-(4-carbamoyl-2-methylphenyl)-1H-pyrrol-2-yl)propanoic acid; 3-(5-(5-bromothiophen-2-yl)-1-(2-methyl-4-(methylsulfonamido)phenyl)-1H -pyrrol-2-yl)propanoic acid; 3-(1-(4-acetamido-2-methylphenyl)-5-(4-(2-methyl-1H-imidazol-1-yl)phenyl)-1H-pyrrol-2-yl)propanoic acid; 3-(5-(4-chloro-2-methoxyphenyl)-1-(2-methyl-4-(methylsulfonamido)phenyl)-1H-pyrrol-2-yl)propanoic acid; 3-(1-(4-carbamoyl-2-methylphenyl)-5-(5-(2-ethyl-1H-imidazoll-yl)thiophen-2-yl)-1H-pyrrol-2-yl)propanoic acid; 3-(1-(4-carbamoyl-2-methylphenyl)-5-(4-chloro-2-formamidophenyl)-1H-pyrrol-2-yl)propanoic acid; 3-(1-(4-carbamoyl-2-methylphenyl)-5-(3-chlorothiophen-2-yl)-1H-pyrrol-2-yl)propanoic acid; 3-(1-(4-carbamoyl-2-methylphenyl)-5-(4-formamido-2-methoxyphenyl)-1H-pyrrol-2-yl)propanoic acid; 3-(5-(3-bromo-5-methoxythiophen-2-yl)-1-(4-carbamoyl-2-methylphenyl)-1H-pyrrol-2-yl)propanoic acid; 3-(1-(4-carbamoyl-2-methylphenyl)-5-(5-(4-hydroxyphenyl)thiophen-2-yl)-1H-pyrrol-2-yl)propanoic acid; 3-(5-(4-bromothiophen-2-yl)-1-(2-methyl-4-(methylsulfonamido)phenyl)-1H-pyrrol-2-yl)propanoic acid; 2-(1-(4-carbamoyl-2-methylphenyl)-5-(4-(thiophen-3-yl)phenyl)-1H-pyrrol-2-yl)acetic acid; 3-(5-(4-bromophenyl)-1-(2-methyl-4-(methylsulfonamido)phenyl)-1H-pyrrol-2-yl)propanoic acid; 3-(5-(5-(1H-imidazol-1-yl)furan-2-yl)-1-(4-carbamoyl-2-methylphenyl)-1H-pyrrol-2-yl)propanoic acid; 3-(5-(4-chloro-2-methoxyphenyl)-1-(2-methyl-4-sulfamoylphenyl)-1H-pyrrol-2-yl)propanoic acid; 3-(1-(4-carbamoyl-2-methylphenyl)-5-(4-chlorothiophen-2-yl)-1H-pyrrol-2-yl)propanoic acid; 3-(5-(5-bromo-4-chlorothiophen-2-yl)-1-(4-carbamoyl-2-methylphenyl)-1H-pyrrol-2-yl)propanoic acid; 3-(5-(4-bromothiophen-2-yl)-1-(2-methyl-4-sulfamoylphenyl)-1H-pyrrol-2-yl)propanoic acid; 3-(5-(4-methoxyphenyl)-1-(2-methyl-4-(pyridin-4-ylcarbamoyl)phenyl)-1H-pyrrol-2-yl)propanoic acid; 3-(1-(4-carbamoyl-2-methylphenyl)-5-(5-(2-methyl-1H-imidazol-1-yl)furan-2-yl)-1H-pyrrol-2-yl)propanoic acid; 3-(5-(5-bromofuran-2-yl)-I-(4-carbamoyl-2-methylphenyl)-1H-pyrrol-2-yl)propanoic acid; 3-(5-(5-(2-methyl-1H-imidazol-1-yl)thiophen-2-yl)-1-(2-methyl-4-sulfamoylphenyl)-1H-pyrrol-2-yl)propanoic acid; 3-(5-(4-bromophenyl)-1-(2-methyl-4-(sulfamoylamino)phenyl)-1H-pyrrol-2-yl)propanoic acid; 3-(5-(4-methoxyphenyl)-1-(2-methyl-4-(sulfamoylamino)phenyl)-1H-pyrrol-2-yl)propanoic acid; 3-(5-(4-chloro-2-methoxyphenyl)-1-(2-methyl-4-(sulfamoylamino)phenyl)-1H-pyrrol-2-yl)propanoic acid; and 3-(1-(4-carbamoyl-2-methylphenyl)-5-(4-methoxyphenyl)-1H-pyrrol-2-yl)butanoic acid.

A second group of inhibitory compounds disclosed in WO 2010/019903 are those of formula II:

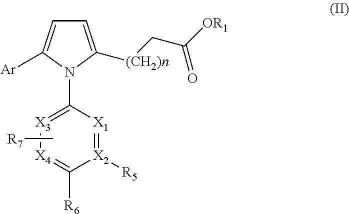

wherein
Ar is selected from the group consisting of aryl, substituted aryl, heteroaryl and substituted heteroaryl;
R₁ is selected from the group consisting of hydrogen, C₁-C₆ alkyl, C₃-C₆ cycloalkyl, aryl, substituted aryl, heteroaryl, and substituted heteroaryl;
X₁-X₄ are independently selected from the group consisting of C and N;
R₅ is selected from the group consisting of hydrogen, hydroxyl, halogen, C₁-C₆ alkyl, C₃-C₆ cycloalkyl, cyano, nitro, carbamoyl, C₁-C₆ alkylcarbamoyl, carboxyl, C₁-C₆ alkylhydroxy, sulfamoyl, C₁-C₆ alkylsulfamoyl, amino, C₁-C₆ alkylamino, C₁-C₆ dialkylamino, C₁-C₆ alkoxyl, and C₃-C₆ cycloalkoxyl;
R₆ is selected from the group consisting of halogen, hydroxyl, carbamoyl, substituted carbamoyl,C₁-C₆ alkylcarbamoyl, sulfamoyl, C₁-C₆ alkylsulfamoyl, C₁-C₆ alkyl, C₃-C₆ cycloalkyl, cyano, nitro, amino, CF₃, carboxyl, ureido, sulfamoylamino, 2-amino-2-oxoethyl, C₁-C₆ alkylamino, C₁-C₆ dialkylamino, arylamino, heteroarylamino, C₁-C₆ alkoxyl, C₃-C₆ cycloalkoxyl, 1H-imidazoll-yl, and 2-methyl-1H-imidazol-1-yl;
or alternatively R₅ and R₆ together form a substituted or unsubstituted 5 or 6 membered heterocyclic ring containing up to 2 heteroatoms chosen from O, N or S;
R₇ is selected from the group consisting of hydrogen, hydroxyl, halogen, C₁-C₆ alkyl, C₃-C₆ cycloalkyl, cyano, nitro, carbamoyl, C₁-C₆ alkylcarbamoyl, sulfamoyl, C₁-C₆ alkylsulfamoyl, amino, C₁-C₆ alkylamino, C₁-C₆ dialkylamino, C₁-C₆ alkoxyl, and C₃-C₆ cycloalkoxyl;
n is 0-3;
wherein R₅ can be hydrogen only when at least one of X₁-X₄ is N;
and further wherein, X₁ and X₃ must be CH or N unless R₅ and R₆ together form a 5 or 6 membered heterocyclic ring or at least one of X₁-X₄ is N;
and wherein Ar excludes substituted or unsubstituted indazol-3-yl and substituted or unsubstituted 1H-pyrazolo[3,4-b]pyridyl;
with the provisos:
proviso IIa: when all X are C and one of R₅, R₆, or R₇ are carboxyl, methylsulfonamido, or sulfamoyl, then Ar cannot be phenyl, substituted or unsubstituted phenyl-(C₁-C₃)-alkyl, or phenyl-(C₂ C₆)-alkenyl; and
proviso IIb: when all X are C and one of R₅, R₆, or R₇ are independently selected from the group consisting of halogen or methyl, then Ar cannot be 4-hydroxyphenyl or 4-C₁-C6 acyl-oxyphenyl; and
proviso IIc: when all X are C and R₅ is methyl, Cl, hydroxyl, trifluoromethyl and R₆ is methyl, methyoxy, carboxyethyl, Cl, or carboxypropyl, and n is 1, then Ar cannot be phenyl, 4-methylphenyl, 4-methoxy-phenyl, 4-halo-phenyl, or thiophen-yl; and
proviso IId: when all X are C and R₅ and R₆ together form a heterocyclic ring and the resulting bicyclic ring system is benzo[d]thiazol substituted at the 2 position with C₁-C₃ alkyl, then Ar cannot be phenyl, 4-F-phenyl, 4-methoxy-phenyl, or thiophen-yl.

Specific compounds within the scope of formula II of WO 2010/019903 are: 3-(5-(4-methoxyphenyl)-1-(1-oxoisoindolin-5-yl)-1H-pyrrol-2-yl)propanoic acid; 3-(1-(4-carbamoyl-3-methoxyphenyl)-5-(4-methoxyphenyl)-1H-pyrrol-2-yl)propanoic acid; 3-(1-(1H-benzo[d]imidazol-6-yl)-5-(4-methoxyphenyl)-1H-pyrrol-2-yl)propanoic acid; 3-(1-(4-carbamoyl-3-hydroxyphenyl)-5-(4-methoxyphenyl)-1H-pyrrol-2-yl)propanoic acid; 3-(1-(5-carbamoylpyridin-2-yl)-5-(4-methoxyphenyl)-1H-pyrrol-2-yl)propanoic acid; 3-(1-(3-chloro-4-hydroxyphenyl)-5-(4-methoxyphenyl)-1H-pyrrol-2-yl)propanoic acid; 3-(1-(6-carbamoylpyridin-3-yl)-5-(4-methoxyphenyl)-1H -pyrrol-2-yl)propanoic acid; 3-(5-(4-methoxyphenyl)-1-(1-oxo-1,3-dihydroisobenzofuran-5-yl)-1H-pyrrol-2-yl)propanoic acid; 3-(1-(1H-benzo[d]imidazol-6-yl)-5-phenyl-1H-pyrrol-2-yl)propanoic acid; 3-(1-(1,3-dioxoisoindolin-5-yl)-5-(4-methoxyphenyl)-1H -pyrrol-2-yl)propanoic acid; 3-(1-(IH-indazol-5-yl)-5-(4-methoxyphenyl)-1H-pyrrol-2-yl)propanoic acid; 3-(5-(4-methoxyphenyl)-1-(6-oxo-1,6-dihydropyridin-3-yl)-1H-pyrrol-2-yl)propanoic acid; 3-(1-(2-aminobenzo[d]thiazol-6-yl)-5-(4-methoxyphenyl)-1H-pyrrol-2-yl)propanoic acid; 3-(1-(4-hydroxy-3-methylphenyl)-5-(4-methoxyphenyl)-1H-pyrrol-2-yl)propanoic acid; 3-(1-(1H-indazol-6-yl)-5-(4-methoxyphenyl)-1H-pyrrol-2-yl)propanoic acid; 3-(1-(2-oxo-2,3-dihydro-1H-benzo[d]imidazol-5-yl)-5-phenyl-1H-pyrrol-2-yl)propanoic acid; 3-(5-(4-methoxyphenyl)-1-(2-oxo-2,3-dihydro-1H-benzo[d]imidazol-5-yl)-1H-pyrrol-2-yl)propanoic acid; 3-(5-(4-methoxyphenyl)-1-(quinolin-6-yl)-1H-pyrrol-2-yl)propanoic acid; 3-(1-(4-hydroxy-3,5-dimethylphenyl)-5-(4-methoxyphenyl)-1H-pyrrol-2-yl)propanoic acid; 3-(5-(4-(1H-imidazol-1-yl)phenyl)-1-(5-(1H-imidazol-1yl) pyridin-2-yl)-1H-pyrrol-2-yl)propanoic acid; 3-(5-(4-(1H-imidazol-1-yl)phenyl)-1-(5-methyl-1H-benzo[d]imidazol-6-yl)-1H-pyrrol-2-yl)propanoic acid; 3-(1-(3-chloro-4-cyanophenyl)-5-(4-methoxyphenyl)-1H-pyrrol-2-yl)propanoic acid; 3-(1-(4-carbamoyl-3-chlorophenyl)-5-(4-methoxyphenyl)-1H-pyrrol-2-yl)propanoic acid; 3-(1-(isoquinolin-6-yl)-5-(4-methoxyphenyl)-1H-pyrrol-2-yl)propanoic acid; 3-(1-(isoquinolin-7-yl)-5-(4-methoxyphenyl)-1H-pyrrol-2-yl)propanoic acid; 3-(1-(1H-indol-5-yl)-5-(4-methoxyphenyl)-1H-pyrrol-2-yl)propanoic acid; 3-(1-(2-oxoindolin-5-yl)-5-phenyl-1H-pyrrol-2-yl)propanoic acid; 3-(1-(3-cyano-4-fluorophenyl)-5-(4-methoxyphenyl)-1H-pyrrol-2-yl)propanoic acid; 3-(1-(4-hydroxy-3-(methoxycarbonyl)phenyl)-5-phenyl-1H-pyrrol-2-yl)propanoic acid; 3-(1-(4-hydroxy-3-(methoxycarbonyl)phenyl)-5-(4-methoxyphenyl)-1H-pyrrol-2-yl)propanoic acid; 3-(1-(3-fluoro-4-hydroxyphenyl)-5-(4-methoxyphenyl)-1H-pyrrol-2-yl)propanoic acid; 3-(1-(4-hydroxy-3-(hydroxymethyl)phenyl)-5-phenyl-1H-pyrrol-2-yl)propanoic acid; 3-(1-(3-aminobenzo[d]isoxazol-6-yl)-5-(4-methoxyphenyl)-1H-pyrrol-2-yl)propanoic acid; 5-(2-(2-carboxyethyl)-5-phenyl-1H-pyrrol-1-yl)-2-hydroxybenzoic acid; 3-(1-(3-aminobenzo[d]isoxazol-5-yl)-5-(4-methoxyphenyl)-1H-pyrrol-2-yl)propanoic acid; 3-(1-(4-hydroxy-3-(hydroxymethyl)phenyl)-5-(4-methoxyphenyl)-1H-pyrrol-2-yl)propanoic acid; 3-(5-(4-(2-methyl-1H-imidazol-1-yl)phenyl)-1-(2-oxo-2,3-dihydrobenzo[d]oxazol-5-yl)-1H-pyrrol-2-yl)propanoic acid; and 3-(5-(4-(2-methyl-1H-imidazol-1-yl)phenyl)-1-(2-oxo-2,3-dihydrobenzo[d]oxazol-6-yl)-1H-pyrrol-2-yl)propanoic acid.

A third group of inhibitory compounds disclosed in WO 2010/019903 are those of formula III:

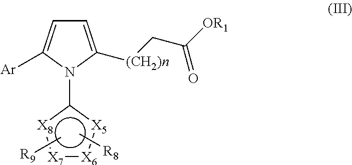

wherein:
Ar is selected from the group consisting of aryl, substituted aryl, heteroaryl and substituted heteroaryl;
R₁ is selected from the group consisting of hydrogen, C₁-C₆ alkyl, C₃-C₆ cycloalkyl, aryl, substituted aryl, heteroaryl, and substituted heteroaryl;
X₅-X₈ are independently selected from the group consisting of N, C and S, wherein at least one X must be Nor S;
R₈ and R₉ are independently selected from the group consisting of hydrogen, halogen, hydroxyl, carbamoyl, substituted carbamoyl, sulfamoyl, substituted sulfamoyl, C₁-C₆ alkyl, C₃-C₆ cycloalkyl, cyano, nitro, amino, CF₃, carboxyl, ureido, sulfamoylamino, 2-amino-2-oxoethyl, C₁-C₆ alkylamino, C₁-C₆ dialkylamino, arylamino, heteroarylamino, C₁-C₆ alkoxyl, C₃-C₆ cycloalkoxyl, aryl, aryloxyl, substituted aryl, heteroaryl, substituted heteroaryl, and null if X₅-X₈ are all N;
n is 0-3;
and wherein Ar excludes substituted or unsubstituted indazol-3-yl and substituted or unsubstituted 1H-pyrazolo[3,4-b]pyridyl.

Specific compounds within the scope of formula III of WO 2010/019903 are: 3-(1-(5-carbamoylthiophen-2-yl)-5-(4-methoxyphenyl)-1H -pyrrol-2-yl)propanoic acid; 3-(1-(5-carbamoylthiazol-2-yl)-5-(4-methoxyphenyl)-1H-pyrrol-2-yl)propanoic acid; 3-(1-(4-carbamoylthiophen-2-yl)-5-(4-methoxyphenyl)-1H -pyrrol-2-yl)propanoic acid; 3-(1-(5-carbamoylthiophen-3-yl)-5-(4-methoxyphenyl)-1H -pyrrol-2-yl)propanoic acid; 3-(1-(4-(2-amino-2-oxoethyl)thiazol-2-yl)-5-(4-methoxyphenyl)-1H-pyrrol-2-yl)propanoic acid; and 3-(1-(4-carbamoylthiazol-2-yl)-5-(4-methoxyphenyl)-1H-pyrrol-2-yl)propanoic acid.

A fourth group of inhibitory compounds disclosed in WO 2010/019903 are those of formula IV:

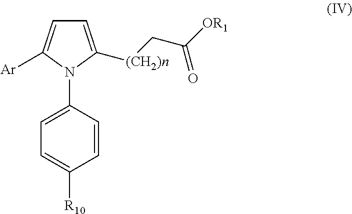

wherein:
Ar is selected from the group consisting of aryl, substituted aryl, heteroaryl and substituted heteroaryl;
R₁ is selected from the group consisting of hydrogen, C₁-C₆ alkyl, C₃-C₆ cycloalkyl, aryl, substituted aryl, heteroaryl, and substituted heteroaryl;
R₁₀ is hydroxyl, carbamoyl, ureido, sulfamoylamino, 2-amino-2-oxoethyl, acetyl, hydroxyethyl, pyridin-3-ylamino, piridin-4-ylamino, 1-methylpyrrolidin-3-yloxy, trifluoroacetyl, imidazol-1-yl, acetamido, methylsulfamido, 2-oxooxazolidin-3-yl, 2-hydroxyethylamino, and methylcarbamoyl;
n is 0-3;
wherein Ar excludes substituted or unsubstituted indazol-3-yl and substituted or unsubstituted 1H-pyrazolo[3,4-b]pyridyl;
with the following proviso:
proviso IVa: when R₁₀ is hydroxyl or carbamoyl, Ar cannot be phenyl, 4-methyl-phenyl, 4-methoxy-phenyl, 4-halo-phenyl, or thiophen-yl when n is 0 or 1.

Specific compounds within the scope of formula IV of WO 2010/019903 are: 3-(1-(4-carbamoylphenyl)-5-(3-methoxyphenyl)-1H-pyrrol-2-yl)propanoic acid; 3-(5-phenyl-1-(4-ureidophenyl)-1H-pyrrol-2-yl)propanoic acid; 3-(5-phenyl-1-(4-(sulfamoylamino)phenyl)-1H-pyrrol-2-yl)propanoic acid; 3-(1-(4-(2-amino-2-oxoethyl)phenyl)-5-phenyl-1H-pyrrol-2-yl)propanoic acid; 3-(1-(4-(2-amino-2-oxoethyl)phenyl)-5-(4-methoxyphenyl)-1H-pyrrol-2-yl)propanoic acid; 3-(5-(4-methoxyphenyl)-1-(4-(methylcarbamoyl)phenyl)-1H-pyrrol-2-yl)propanoic acid; 3-(1-(4-hydroxyphenyl)-5-(4-(2-methyl-1H-imidazol-1-yl)phenyl)-1H-pyrrol-2-yl)propanoic acid; 2-(1-(4-hydroxyphenyl)-5-(4-methoxyphenyl)-1H-pyrrol-2-yl)acetic acid; 3-(5-(4-(1H-imidazol-1-yl)phenyl)-1-(4-hydroxyphenyl)-1H-pyrrol-2-yl)propanoic acid; 3-(5-(5-(1H-imidazol-1-yl)thiophen-2-yl)-1-(4-hydroxyphenyl)-1H-pyrrol-2-yl)propanoic acid; 3-(1-(4-acetylphenyl)-5-(4-methoxyphenyl)-1H-pyrrol-2-yl)propanoic acid; 3-(1-(4-(1-hydroxyethyl)phenyl)-5-phenyl-1H-pyrrol-2-yl)propanoic acid; 3-(1-(4-hydroxyphenyl)-5-(4-(2-methyl-1H-imidazol-1-yl)thiophen-2-yl)-1H-pyrrol-2-yl)propanoic acid; 3-(5-(4-methoxyphenyl)-1-(4-(pyridin-3-ylamino)phenyl)-1H-pyrrol-2-yl)propanoic acid; 3-(5-(4-methoxyphenyl)-1-(4-(1-methylpyrrolidin-3-yloxy)phenyl)-1H-pyrrol-2-yl)propanoic acid; 3-(5-(4-methoxyphenyl)-1-(4-(pyridin-4-ylamino)phenyl)-1H-pyrrol-2-yl)propanoic acid; 3-(5-phenyl-1-(4-(2,2,2-trifluoroacetyl)phenyl)-1H-pyrrol-2-yl)propanoic acid; 3-(5-(4-methoxyphenyl)-1-(4-(2,2,2-trifluoroacetyl)phenyl)-1H-pyrrol-2-yl)propanoic acid; 3-(1-(4-(1H-imidazol-1-yl)phenyl)-5-(4-methoxyphenyl)-1H-pyrrol-2-yl)propanoic acid; 3-(5-(4-chloro-2-methoxyphenyl)-1-(4-hydroxyphenyl)-1H-pyrrol-2-yl)propanoic acid; 3-(1-(4-acetamidophenyl)-5-(4-chloro-2-methoxyphenyl)-1H-pyrrol-2-yl)propanoic acid; 3-(1-(4-carbamoylphenyl)-5-(4-chloro-2-methoxyphenyl)-1H-pyrrol-2-yl)propanoic acid; 3-(5-(4-chloro-2-methoxyphenyl)-1-(4-(methylsulfonamido)phenyl)-1H-pyrrol-2-yl)propanoic acid; 3-(5-(4-(2-methyl-1H-imidazol-1yl)phenyl)-1-(4-(2-oxooxazolidin-3-yl)phenyl)-1H-pyrrol-2-yl)propanoic acid; 3-(5-(4-(2-methyl-1H-imidazol-1-yl)thiophen-2-yl)-1-(4-(methylsulfonamido)phenyl)-1H-pyrrol-2-yl)propanoic acid; 3-(1-(4-acetamidophenyl)-5-(4-(2-methyl-1H-imidazol-1-yl)phenyl)-1H-pyrrol-2-yl)propanoic acid; 3-(5-(2-methoxy-4-(2-methyl-1H-imidazol-1-yl)phenyl)-1-(4-(methylsulfonamido)phenyl)-1H-pyrrol-2-yl)propanoic acid; 3-(1-(4-acetamidophenyl)-5-(4-(2-methyl-1H-imidazol-1-yl)thiophen-2-yl)-1H-pyrrol-2-yl)propanoic acid; 3-(5-(4-(2-methyl-1H-imidazol-1-yl)phenyl)-1-(4-(methylsulfonamido)phenyl)-1H-pyrrol-2-yl)propanoic acid; 3-(1-(4-(2-hydroxyethylamino)phenyl)-5-(4-(2-methyl-1H-imidazol-1-yl)phenyl)-1H-pyrrol-2-yl)propanoic acid; and 3-(1-(4-hydroxyphenyl)-5-(5-(2-methyl-1H-imidazol-1-yl)thiophen-2-yl)-1H-pyrrol-2-yl)propanoic acid.

Inhibitors of S-nitrosoglutathione reductase disclosed in WO 2010/019910 include compounds of formula I below:

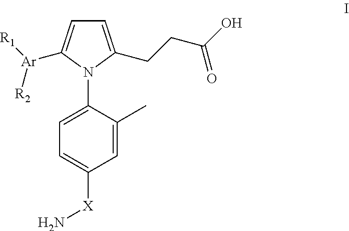

wherein:
Ar is selected from the group consisting of phenyl and thiophen-yl;
R₁ is selected from the group consisting of unsubstituted imidazolyl, substituted imidazolyl, chloro, bromo, fluoro, hydroxy, and methoxy;
R₂ is selected from the group consisting of hydrogen, methyl, chloro, fluoro, hydroxy, methoxy, ethoxy, propoxy, carbamoyl, dimethylamino, amino, formamido, and trifluoromethyl; and
X is selected from the group consisting of CO and SO₂.

Specific compounds within the scope of formula I of WO 2010/019910 include: 3-(5-(4-(1H-imidazol-1-yl)phenyl)-1-(4-carbamoyl-2-methylphenyl)-1H-pyrrol-2-yl)propanoic acid; 3-(5-(5-(1H-imidazol-1-yl)thiophen-2-yl)-1-(4-carbamoyl-2-methylphenyl)-1H-pyrrol-2-yl)propanoic acid; 3-(1-(4-carbamoyl-2-methylphenyl)-5-(4-(2-methyl-1H-imidazol-1-yl)phenyl)-1H-pyrrol-2-yl)propanoic acid; 3-(1-(4-carbamoyl-2-methylphenyl)-5-(4-(4-methyl-1H-imidazol-1-yl)phenyl)-1H-pyrrol-2-yl)propanoic acid; 3-(1-(4-carbamoyl-2-methylphenyl)-5-(4-(2-ethyl-1H-imidazol-1-yl)phenyl)-1H-pyrrol-2-yl)propanoic acid;3-(5-(4-(1H-imidazol-1-yl)thiophen-2-yl)-1-(4-carbamoyl-2-methylphenyl)-1H-pyrrol-2-yl)propanoic acid;3-(1-(4-carbamoyl-2-methylphenyl)-5-(5-(2-methyl-1H-imidazol-1-yl)thiophen-2-yl)-1H-pyrrol-2-yl)propanoic acid; 3-(1-(4-carbamoyl-2-methylphenyl)-5-(3-fluoro-4-(1H-imidazol-1-yl)phenyl)-1H-pyrrol-2-yl)propanoic acid; 3-(1-(4-carbamoyl-2-methylphenyl)-5-(3-fluoro-4-(2-methyl-1H-imidazol-1-yl)phenyl)-1H-pyrrol-2-yl)propanoic acid; 3-(1-(4-carbamoyl-2-methylphenyl)-5-(4-(2-methyl-1H-imidazol-1-yl)thiophen-2-yl)-1H-pyrrol-2-yl)propanoic acid; 3-(1-(4-carbamoyl-2-methylphenyl)-5-(2-methoxy-4-(2-methyl-1H-imidazol-1-yl)phenyl)-1H-pyrrol-2-yl)propanoic acid;3-(5-(4-(1H-imidazol-1-yl)-2-methoxyphenyl)-1-(4-carbamoyl-2-methylphenyl)-1H-pyrrol-2-yl)propanoic acid; 3-(1-(4-carbamoyl-2-methylphenyl)-5-(5-(2-methyl-1H-imidazol-1-yl)thiophen-3-yl)-1H-pyrrol-2-yl)propanoic acid; 3-(1-(4-carbamoyl-2-methylphenyl)-5-(5-(2-ethyl-1H-imidazol-1-yl)thiophen-2-yl)-1H-pyrrol-2-yl)propanoic acid; and 3-(5-(5-(2-methyl-1H-imidazol-1-yl)thiophen-2-yl)-1-(2-methyl-4-sulfamoylphenyl)-1H-pyrrol-2-yl)propanoic acid.

Additional compounds include: 3-(1-(4-carbamoyl-2-methylphenyl)-5-(4-hydroxyphenyl)-1H-pyrrol-2-yl)propanoic acid; 3-(5-(5-bromothiophen-2-yl)-1-(4-carbamoyl-2-methylphenyl)-1H -pyrrol-2-yl)propanoic acid; 3-(1-(4-carbamoyl-2-methylphenyl)-5-(4-methoxyphenyl)-1H-pyrrol-2-yl)propanoic acid; 3-(5-(4-bromophenyl)-1-(4-carbamoyl-2-methylphenyl)-1H -pyrrol-2-yl)propanoic acid; 3-(1-(4-carbamoyl-2-methylphenyl)-5-(3-chloro-4-methoxyphenyl)-1H -pyrrol-2-yl)propanoic acid; 3-(1-(4-carbamoyl-2-methylphenyl)-5-(3-fluoro-4-methoxyphenyl)-1H-pyrrol-2-yl)propanoic acid; 3-(1-(4-carbamoyl-2-methylphenyl)-5-(3-chloro-4-hydroxyphenyl)-1H -pyrrol-2-yl)propanoic acid; 3-(1-(4-carbamoyl-2-methylphenyl)-5-(4-methoxy-3-methylphenyl)-1H -pyrrol-2-yl)propanoic acid; 3-(1-(4-carbamoyl-2-methylphenyl)-5-(3-methoxyphenyl)-1H -pyrrol-2-yl)propanoic acid; 3-(5-(4-amino-3-chlorophenyl)-1-(4-carbamoyl-2-methylphenyl)-1H-pyrrol-2-yl)propanoic acid; 3-(1-(4-carbamoyl-2-methylphenyl)-5-(3,4-difluorophenyl)-1H -pyrrol-2-yl)propanoic acid; 3-(1-(4-carbamoyl-2-methylphenyl)-5-(2,4-difluorophenyl)-1H -pyrrol-2-yl)propanoic acid; 3-(1-(4-carbamoyl-2-methylphenyl)-5-(4-chlorophenyl)-1H -pyrrol-2-yl)propanoic acid; 3-(5-(4-bromothiophen-2-yl)-1-(4-carbamoyl-2-methylphenyl)-1H-pyrrol-2-yl)propanoic acid; 3-(1-(4-carbamoyl-2-methylphenyl)-5-(4-fluoro-3-methoxyphenyl)-1H -pyrrol-2-yl)propanoic acid; 3-(1-(4-carbamoyl-2-methylphenyl)-5-(4-carbamoyl-3-fluorophenyl)-1H-pyrrol-2-yl)propanoic acid; 3-(1-(4-carbamoyl-2-methylphenyl)-5-(4-methoxy-2-methylphenyl)-1H-pyrrol-2-yl)propanoic acid; 3-(1-(4-carbamoyl-2-methylphenyl)-5-(4-chloro-2-fluorophenyl)-1H-pyrrol-2-yl)propanoic acid; 3-(1-(4-carbamoyl-2-methylphenyl)-5-(4-fluorophenyl)-1H-pyrrol-2-yl)propanoic acid; 3-(1-(4-carbamoyl-2-methylphenyl)-5-(4-fluoro-2-methylphenyl)-1H-pyrrol-2-yl)propanoic acid; 3-(1-(4-carbamoyl-2-methylphenyl)-5-(4-chloro-2-methoxyphenyl)-1H-pyrrol-2-yl)propanoic acid; 3-(1-(4-carbamoyl-2-methylphenyl)-5-(2-chloro-4-methoxyphenyl)-1H -pyrrol-2-yl)propanoic acid; 3-(1-(4-carbamoyl-2-methylphenyl)-5-(2-ethoxy-4-fluorophenyl)-1H -pyrrol-2-yl)propanoic acid; 3-(1-(4-carbamoyl-2-methylphenyl)-5-(4-methoxy-2-(trifluoromethyl)phenyl)-1H-pyrrol-2-yl)propanoic acid; 3-(1-(4-carbamoyl-2-methylphenyl)-5-(4-fluoro-2-methoxyphenyl)-1H -pyrrol-2-yl)propanoic acid; 3-(1-(4-carbamoyl-2-methylphenyl)-5-(4-chloro-3-fluorophenyl)-1H-pyrrol-2-yl)propanoic acid; 3-(1-(4-carbamoyl-2-methylphenyl)-5-(4-chloro-2-ethoxyphenyl)-1H-pyrrol-2-yl)propanoic acid; 3-(5-(5-bromo-2-methoxyphenyl)-1-(4-carbamoyl-2-methylphenyl)-1H -pyrrol-2-yl)propanoic acid; 3-(5-(4-bromo-2-methoxyphenyl)-1-(4-carbamoyl-2-methylphenyl)-1H-pyrrol-2-yl)propanoic acid; 3-(1-(4-carbamoyl-2-methylphenyl)-5-(4-chloro-2-hydroxyphenyl)-1H-pyrrol-2-yl)propanoic acid; 3-(5-(5-bromothiophen-3-yl)-1-(4-carbamoyl-2-methylphenyl)-1H -pyrrol-2-yl)propanoic acid; 3-(1-(4-carbamoyl-2-methylphenyl)-5-(4-hydroxy-3-methylphenyl)-1H-pyrrol-2-yl)propanoic acid; 3-(1-(4-carbamoyl-2-methylphenyl)-5-(2-carbamoyl-4-chlorophenyl)-1H-pyrrol-2-yl)propanoic acid; 3-(1-(4-carbamoyl-2-methylphenyl)-5-(2-methoxyphenyl)-1H-pyrrol-2-yl)propanoic acid; 3-(1-(4-carbamoyl-2-methylphenyl)-5-(2,4-dimethoxyphenyl)-1H -pyrrol-2-yl)propanoic acid; 3-(1-(4-carbamoyl-2-methylphenyl)-5-(4-chloro-2-propoxyphenyl)-1H-pyrrol-2-yl)propanoic acid; 3-(1-(4-carbamoyl-2-methylphenyl)-5-(4-hydroxy-2-methoxyphenyl)-1H-pyrrol-2-yl)propanoic acid; 3-(1-(4-carbamoyl-2-methylphenyl)-5-(4-chloro-2-(dimethylamino)phenyl)-1H-pyrrol-2-yl)propanoic acid; 3-(1-(4-carbamoyl-2-methylphenyl)-5-(5-chlorothiophen-2-yl)-1H-pyrrol-2-yl)propanoic acid; 3-(1-(4-carbamoyl-2-methylphenyl)-5-(4-chloro-2-formamidophenyl)-1H-pyrrol-2-yl)propanoic acid; 3-(1-(4-carbamoyl-2-methylphenyl)-5-(3-chlorothiophen-2-yl)-1H-pyrrol-2-yl)propanoic acid; 3-(1-(4-carbamoyl-2-methylphenyl)-5-(4-formamido-2-methoxyphenyl)-1H-pyrrol-2-yl)propanoic acid; 3-(5-(3-bromo-5-methoxythiophen-2-yl)-1-(4-carbamoyl-2-methylphenyl)-1H-pyrrol-2-yl)propanoic acid; 3-(1-(4-carbamoyl-2-methylphenyl)-5-(4-chlorothiophen-2-yl)-1H -pyrrol-2-yl)propanoic acid; 3-(5-(5-bromo-4-chlorothiophen-2-yl)-1-(4-carbamoyl-2-methylphenyl)-1H-pyrrol-2-yl)propanoic acid; and 3-(5-(4-bromothiophen-2-yl)-1-(2-methyl-4-sulfamoylphenyl)-1H-pyrrol-2-yl)propanoic acid.

A second group of inhibitory compounds disclosed in WO 2010/019910 are those of formula II:

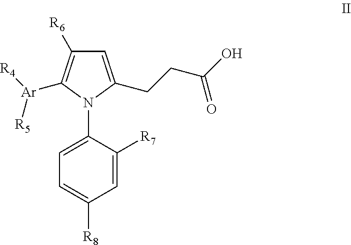

wherein:
Ar is selected from the group consisting of phenyl and thiophen-yl;
R₄ is selected from the group consisting of unsubstituted imidazolyl and substituted imidazolyl;
R₅ is selected from the group consisting of hydrogen, fluoro, hydroxy, and methoxy;
R₆ is selected from the group consisting of hydrogen, chloro, bromo, and fluoro;
R₇ is selected from the group consisting of hydrogen, and methyl; and
R₈ is selected from the group consisting of CONH₂, SO₂NH₂, and NHSO₂CH₃.

Specific compounds within the scope of formula II of WO 2010/019910 include: 3-(5-(5-(2-methyl-1H-imidazol-1-yl)thiophen-2-yl)-I-(4-sulfamoylphenyl)-1H-pyrrol-2-yl)propanoic acid; 3-(5-(5-(2-methyl-1H-imidazol-1-yl)thiophen-2-yl)-1-(2-methyl-4-(methylsulfonamido)phenyl)-1H-pyrrol-2-yl)propanoic acid; 3-(5-(4-(1H-imidazol-1-yl)phenyl)-1-(2-methyl-4-(methylsulfonamido)phenyl)-1H-pyrrol-2-yl)propanoic acid; 3-(5-(4-(2-methyl-1H-imidazol-1-yl)phenyl)-1-(2-methyl-4-(methylsulfonamido)phenyl)-1H-pyrrol-2-yl)propanoic acid; 3-(5-(4-(2-methyl-1H-imidazol-1-yl)thiophen-2-yl)-1-(2-methyl-4-(methylsulfonamido)phenyl)-1H-pyrrol-2-yl)propanoic acid; 3-(5-(5-(2-methyl-1H-imidazol-1-yl)thiophen-2-yl)-1-(2-methyl-4-(methylsulfonamido)phenyl)-1H-pyrrol-2-yl)propanoic acid; 3-(5-(4-(2-methyl-1H-imidazol-1-yl)thiophen-2-yl)-1-(4-(methylsulfonamido)phenyl)-1H-pyrrol-2-yl)propanoic acid; 3-(5-(2-methoxy-4-(2-methyl-1H-imidazol-1-yl)phenyl)-1-(4-(methylsulfonamido)phenyl)-1H-pyrrol-2-yl)propanoic acid; and 3-(5-(4-(2-methyl-1H-imidazol-1-yl)phenyl)-1-(4-(methylsulfonamido)phenyl)-1H-pyrrol-2-yl)propanoic acid.

B. Drug Formulation

The compounds described above will typically be administered to patients in a pharmaceutical composition comprising the compound and a pharmaceutically acceptable carrier. The carrier may be any solvent, diluent, liquid or solid vehicle that is pharmaceutically acceptable and typically used in formulating drugs. Guidance concerning the making of pharmaceutical formulations can be obtained from standard works in the art (see, e.g., Remington's Pharmaceutical Sciences, 16^{th} edition, E. W. Martin, Easton, Pa. (1980)). In addition, pharmaceutical compositions may contain any of the excipients that are commonly used in the art. Examples of carriers or excipients that may be present include, but are not limited to, sugars (e.g., lactose, glucose and sucrose); starches, such as corn starch or potato starch; cellulose and its derivatives (e.g., sodium carboxymethyl cellulose, ethyl cellulose, or cellulose acetate); malt; gelatin; talc; cocoa butter; oils (e.g., peanut oil, cottonseed oil, safflower oil, sesame oil, olive oil, corn oil, or soybean oil); glycols; buffering agents; saline; Ringer's solution; alcohols; lubricants; coloring agents; dispersing agents; coating agents; flavoring agents; preservatives; or antioxidants.

The invention is compatible with the delivery of compounds by any route known in the art, including peroral, internal, nasal, lingual, transdermal, intravenous, intra-arterial, intramuscular, intraperitoneal, intracutaneous and subcutaneous routes. The most preferred routes are orally, especially using dosage forms such as tablets, or capsules, and parenterally by infusion or injection. In cases where a compound is susceptible to degradation in the stomach of a patient, parenteral delivery is preferred or the compound may be enterically coated and administered orally.

It will be understood that pharmaceutical compositions may contain any pharmaceutically acceptable form of compound, i.e., any form which maintains therapeutic activity and which does not cause unacceptable adverse effects when administered. For example, a compound may be in the form of a pharmaceutically acceptable salt, ester or pro-drug.

Liquid dosage forms for oral administration include, but are not limited to, pharmaceutically acceptable emulsions, microemulsions, solutions, suspensions, syrups and elixirs. In addition to the active compounds, liquid dosage form may contain inert diluents commonly used in the art, such as, for example, water, or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethyl formamide, oils, glycerol, alcohols, polyethylene glycols, and fatty acid esters.

Injectable preparations may be in the form of sterile, injectable aqueous or oleaginous suspensions, diluents or solvents that may be used may include 1,3-butanediol, water, Ringer's solution and isotonic saline solutions. In addition, oils or fatty acids may be present.

In oral dosage forms such as capsules, tablets, pills, powders or granules, the active compound will typically be mixed with at least one inert, pharmaceutically acceptable excipient or carrier such as sodium citrate, or diacalcium phosphate and/or: fillers or extenders such as starches, lactose, sucrose, glucose, mannitol and silicic acid; binders such as, for example, carboxymethyl cellulose, alginates, gelatin, polyvinylpyrrolidinone, and acacia, humectants such as glycerol; disintegrating agents such as calcium carbonate, silicates or sodium carbonate; solution retarding agents such as paraffin; absorption accelerators such as quaternary ammonium compound; wetting agents such as cetyl alcohol or glycerol monostearate; absorbents such as kaolin; and lubricants, such as talc, magnesium stearate; sodium lauryl sulphate, etc. In addition, dosage forms may include buffering and flavoring agents.

Pharmaceutical compositions will typically be given to a patient in one or more unit dosage forms. A “unit dosage form” refers to a single drug administration entity, e.g., a single tablet, capsule or injection vial. Sufficient compound should be present in a unit dosage form to be therapeutically effective when administered as prescribed. For example, if a patient is to take two tablets, sufficient compound should be present in each tablet so that, when they are taken together, they are therapeutically effective.

As used herein, a “therapeutically effective amount” is an amount sufficient to alleviate at least one symptom associated with acetaminophen-induced liver toxicity or to reduce or prevent the injury to hepatic cells that would occur in the absence of administered drug in a significant number of patients that are risk for liver damage due to the excessive acetaminophen ingestion. Since not all patients respond equally to drugs, a therapeutically effective amount will not always be successful in the treatment of patients. A determination of effectiveness can be made using standard clinical methods for assessing liver function.

C. Treatment Methods

Patients at risk for liver damage due to the excessive ingestion of acetaminophen may be treated by administering one or more of the compounds described above. The exact dosage will depend upon the particular compound being given and will be determined using procedures well known in the art, balancing toxicity and therapeutic efficacy. In general, it is expected that a therapeutically effective amount of compound will be 1 μg-10 g/kg body weight and in most instances 10 μg-1 g/kg body weight, or 10 μg to 100 mg/kg per day. At the discretion of the attending medical personnel, treatment may be repeated for several days, with successful completion typically being evidenced by a subsiding of symptoms and a return of tests of liver function to normal values.

Compounds may be administered as the sole active agents in a dosage form, or they may be combined with other drugs to improve overall effectiveness. In particular the compounds may be given with other treatments for acetaminophen toxicity such as N-acetylcysteine (NAC). The latter is typically administered intravenously to patients that have overdosed acetaminophen. For example, a patient may be infused with 150 mg/kg of NAC in a 200 ml solution given over a period of 60 minutes. Patients may then be infused with a maintenance dose containing lower amounts of NAC. For example, a patient may be given 50/mg/kg in 500 ml given over 4 hours followed by 100 mg/kg in 1000 ml given over 16 hours. These dosages will be adjusted by attending medical personnel based upon individual circumstances and patient characteristics. It is believed that the compound N6547, as well as other inhibitors of S-nitrosoglutathione reductase, may act synergistically to protect the liver from acetaminophen-induced damage, i.e., the drugs may produce a better effect together than when either drug is used alone. Other treatments for liver toxicity, e.g., activated charcoal may also be administered to a patient as well as palliative drugs.

D. Uses

The most obvious use of the present invention is in the administration of compounds to individuals to treat or prevent an acute overdose of acetaminophen. It should be appreciated however that treatments may also involve administering compounds to patients that chronically take acetaminophen, e.g., on a daily basis, in order to prevent long term risk of liver damage. Compounds may also be given as a preventative to patients that that take normal doses of acetaminophen but that may be unusually susceptible to liver damage because of disease, chemotherapy or genetic factors.

### EXAMPLES

A. Summary

In order to identify novel regulators of liver development in vivo, a chemical genetic screen in zebrafish embryos was performed, which revealed that nitric oxide (NO) signaling modulated liver development. The NO donor Deta NONOate caused an increase in liver size at 72 hours post fertilization (hpf), assessed by in situ hybridization for liver fatty acid binding protein (lfabp), whereas embryos exposed to the nitric oxide synthase (NOS) inhibitor L-NAME had smaller livers. NO signaling did not observably affect the size of other organs derived from endoderm. Timecourse studies revealed that NO signaling was most effective at modulating liver size when embryos were treated earlier in development (24-48 hpf) coincident with the budding stage of liver formation. Knockdown of nos1 (constitutive isoform), but not nos2 (inducible isoform), caused a significant decrease in liver size. NO can mediate downstream effects via cGMP signaling in the vasculature or direct modification of protein thiols (S-nitrosylation). S-nitroso-glutathione (GSNO) and L-NAME were able to modulate liver size in cloche embryos (which lack vasculature) suggesting that cGMP-independent (nitrosylation) pathways play a major role in regulating liver development. Accordingly, loss of the enzyme responsible for metabolizing S-nitrosothiols, GSNO reductase (GSNOR), activity resulted in larger livers. In order to assess the role of NO during liver injury, we exposed embryos and adults to acetaminophen (APAP). This caused a decrease in liver size and death, both of which were overcome by co-exposure to NO donors. Coexposure of APAP with L-NAME caused a further decrease in liver size and more extensive loss in survival. In addition, mice carrying a deletion for the GSNOR gene were protected from APAP injury. Treatment of mice with the GSNOR inhibitor improved liver enzyme levels and hepatocyte necrosis after APAP exposure, and was synergistic with the clinical antidote NAC. These data suggest that NO signaling is required for optimal liver development and regeneration following APAP induced liver injury.

B. Methodology and Results

Liver Development in Zebrafish

The liver arises from anterior endodermal progenitor cells. Liver development can be divided into three stages, specification (18-24 hours post fertilization, hpf), budding/differentiation (24-72 hpf), and hepatic outgrowth (72-96 hpf). The various stages of liver development can be observed in the embryo by in situ hybridization for hhex (endodermal progenitors), foxA3 (differentiated endoderm) and liver fatty acid binding protein (lfabp, marker of hepatocytes).

Chemical Screening

In an effort to identify novel regulators of liver formation and homeostasis, a library of 2880 biologically active compounds (obtained from the ICCB at Harvard Medical School) was previously screened for global effects on liver development in zebrafish. Hepatogenesis was assessed in vivo using fluorescent reporter fish where the hepatocyte-specific lfabp promoter drives GFP expression. All embryos were imaged with fluorescence microscopy at 72 hpf, then fixed and subjected to in situ hybridization for lfabp to confirm the in vivo findings. Of the chemical treatments, 191 compounds (6.6%) increased, and 344 (11%) decreased liver formation. Among the compounds screened, the NO precursor L-Arginine and the NO donor Deta caused an increase in liver size, whereas exposure to the NOS inhibitor L-NAME resulted in a small liver.

Effect of NO Signaling on Liver Size During Development

In order to identify regulators of liver formation a chemical genetic screen was conducted. Several of the chemicals identified by the screen were known to impact on NO signaling. Based on these initial observations, well-established modulators of NO signaling were secondarily screened for effects on liver formation. Exposure to the NO precursor, L-Arginine (L-Arg)(10 μM), from 24-72 hpf caused a dramatic increase in liver size, as determined by in situ hybridization for the hepatocyte-specific genes lfabp and transferrin. Whereas, exposure to the NOS inhibitor, L-N^{G}-Nitroarginine methyl ester (L-NAME) (10 μM), from 24-72 hpf caused a decrease in liver size. The contrasting effects of L-Arg and L-NAME on liver formation were confirmed by in vivo fluorescent microscopy of lfabp:GFP transgenic zebrafish. To quantify the phenotypic changes caused by NO signaling, the liver size (region of lfabp expression) of treated embryos were scored as large, medium or small. Phenotypic analysis demonstrated that approximately 51% of the embryos exposed to L-Arg had a large liver; whereas L-NAME treatment diminished liver size in 62% of the embryos examined.

In addition to L-Arg, other NO donors such as Diethylenetriamine-NONOate (Deta) and S-Nitroso-N-Acetyl-D,L-Penicillamine (SNAP) at optimal doses could also increase liver size. Similarly, an alternative NOS inhibitor, NG-amino-L-Arginine (L-NMMA), could decrease liver size in a dose-dependent manner. Importantly, the inactive enantiomer D-Arginine (D-Arg) had no effect on liver size at any dose tested. To confirm that the effects of NO signaling were specific to the liver, the formation of other organ systems was examined in treated embryos by in situ hybridization for foxA3 (pan-endoderm), trypsin (exocrine pancreas), insulin (endocrine pancreas), cmlc2 (heart) and ifabp (gut). With the exception of the liver, visible in the foxA3 expression pattern, there was no discernable change in expression patterns in other organ systems. To demonstrate that L-Arg and L-NAME were modulating NO production in vivo a cell-trappable fluorescent probe, Cu(2)(FL2E), was able to show a distinct increase in NO in the developing endoderm that was inhibited by L-NAME. Flow cytometric analysis of GFP+ cells in treated lfabp:GFP embryos revealed that L-Arg caused an increase in hepatocytes per embryo compared to control, whereas L-NAME caused a decrease. These findings were corroborated by qPCR for lfabp. Together, these studies suggest that NO signaling regulates liver size during development.

NO Signaling Affects Hepatic Progenitor Cell Proliferation

To further characterize the role of NO signaling on liver formation, we studied the temporal requirements for NO signaling. Embryos were exposed to Deta (10 μM), L-Arg (10 μM) or L-NAME (10 μM) from 24-48 or 48-72 hpf at which point liver size was assessed. Chemical modulators of NO signaling caused dramatic changes in liver formation in embryos exposed from 24-48 hpf, whereas, no changes in liver size were observed in embryos treated from 48-72 hpf. Given that NO signaling during the budding phase of liver development (24-48 hpf) was most effective at modulating liver size, the effect of NO signaling on hepatic progenitors in the developing endoderm was examined by in situ hybridization for the early hepatic progenitor/endoderm markers hhex and foxA3. Exposure of embryos to L-Arg (10 μM) caused a subtle yet significant increase in the hepatic bud size at 36 hpf (region of hhex expression) in 26% of embryos, whereas L-NAME diminished hepatic bud size in 33% of embryos. Similarly, examination of the foxA3 expression pattern revealed that L-Arg increased hepatic bud size at 48 hpf in 51% embryos, whereas L-NAME restricted the size of the hepatic bud in 60% of embryos.

The expression pattern of an alternate hepatic progenitor marker prox1 corroborated that chemical modulators of NO signaling alter the size of the developing hepatic bud. The NO donor Deta (10 μM) had a similar effect as L-Arg, increasing the region of hhex and foxA3 expression in the developing hepatic bud. To determine whether the increase in hepatic bud size was due to cell proliferation, we examined BrdU incorporation and found that L-Arg caused an increase in BrdU positive cells, whereas L-NAME reduced the number of BrdU positive cells. To confirm the proliferative effect of NO signaling, treated embryos were stained for the mitotic marker pH3 and this corroborated that L-Arg increased hepatic proliferation, whereas L-NAME inhibited cell cycle progression. These findings were verified by qPCR for hhex and foxA3. Together, these data suggest that NO signaling enhances proliferation of hepatic progenitors and regulates hepatic bud size during development.

Effect of NO Signaling on Endodermal Organs Other than the Liver

In order to examine the effect that NO signaling had on other endodermal organs including the pancreas and gut, embryos were exposed to Deta (10 μM) or L-NAME (10 μM) from 18 hpf until 72 hpf. Endodermal derived tissue, the exocrine pancreas and gut were stained by in situ hybridization for foxa3, trypsin and ifabp, respectively. Despite the fact that NO signaling modulated liver size, exposure to the NO donor Deta or the NOS inhibitor L-NAME did not observably affect the size of other endodermal organs.

Nos 1 is Required for Optimal Liver Development

To clarify the role of NO signaling in liver development, we took a genetic approach, using previously validated morpholinos (MO) to knockdown the expression of nos1 (constitutive isoform) and nos2 (inducible isoform). Knockdown of nos1 had a dramatic effect inhibiting liver formation as determined by lfabp expression, while knockdown of nos2 had no effect. Splice-site targeted MOs against nos1 also inhibited liver formation. Phenotypic analysis demonstrated that 75% of nos1 ATG morphants and 64% of nosl splice morphants displayed a small liver. The effect of nosl knockdown on liver formation was confirmed in vivo by fluorescent microscopy of lfabp:GFP transgenic zebrafish. Flow cytometric analysis in lfabp:GFP morphants revealed that knockdown of nos1 caused a decrease in the number of hepatocytes per embryo compared to uninjected controls. Examination of foxA3 expression demonstrated that knockdown of nos1, but not nos2, caused a dramatic reduction in hepatic bud size. Together, these results suggest that nos1 (cNOS) is required for optimal liver formation.

S-Nitrosothiol Signaling Regulates Liver Formation Independently of cGMP

NO signaling can occur via 2 distinct mechanisms namely, cGMP-dependent vasodilation and S-nitrosylation pathways. In order to clarify the importance of the well-known vascular effects of NO, we examined the effect of chemical modulators of NO signaling in cloche mutants that have no endothelial cells and therefore lack any vasculature. Suprisingly, exposure to L-Arg (10 μM) could still increase liver size in 37% of treated embryos, whereas 40% of L-NAME (10 μM) treated cloche embryos had a small liver. Similarly, the S-nitrosothiol S-nitrosoglutathione (GSNO) (5 μM) increased liver size in 46% of treated cloche embryos. These studies suggest that a vasculature is not necessary for NO to affect liver development. To further clarify which downstream pathways were involved in liver formation, embryos were exposed to the cGMP analogue (8BrGMP, 10 mM) and this had no effect on liver size, however, the S-nitrosothiol GSNO (5 μM) enhanced liver size in 40% of embryos. Together, these results suggest that NO signaling can regulate liver size by S-nitrosothiol signaling independently of cGMP-dependent effects on the vasculature.

S-nitrosoglutathione reductase (GSNOR) is the major enzyme responsible for metabolizing S-nitrosothiols and is therefore a key negative regulator of S-nitrosylation pathways. We reasoned that by inhibiting GSNOR function we could stimulate S-nitrosothiol signaling and modulate liver formation. Embryos exposed to the GSNOR inhibitor N6547 (GSNORi, 1 μM) from 24-72 hpf displayed a dramatic increase in liver size in 40% of the embryos examined. This finding was corroborated by knocking down GSNOR expression with an ATG MO, which caused 51% of the morphants to have an enlarged liver. Analysis of GSNORi treated lfabp:GFP embryos by flow cytometry showed that there was an increase in the number of hepatocytes per embryo compared to controls. The positive effects of GSNORi on hepatic growth during development were verified by qPCR for lfabp. Consistent with our earlier findings, embryos exposed to GSNORi from 24 to 48 hpf exhibited a distinct increase in hepatic bud size (sid4 expression). Together, these findings suggest that GSNOR inhibition can enhance S-nitrosothiol signaling to promote proliferation in the developing liver.

GSNORi and NAC Are Synergistically Hepatoprotective Following APAP Injury in Zebrafish Larvae

In order to examine the possible hepatoprotective effect of GSNORi in the context of acetaminophen (APAP) induced liver injury. Transgenic lfabp:GFP embryos were exposed to a sub-lethal dose of APAP (5 mM) from 48 to 60 hpf followed by treatment during the recovery phase (60-96 hpf) with L-NAME, GSNORi and/or NAC. APAP exposure caused a dramatic decrease in liver size and this decrease was exacerbated in embryos treated with L-NAME (10 μM) during the recovery phase. In contrast, the clinical antidote N-acetylcysteine (NAC, 10 μM) was able to enhance liver size in APAP exposed embryos. Importantly, GSNORi (1 μM) exposure increased liver size following APAP exposure and it synergized with NAC to further promote hepatic regeneration. In order to further investigate the proliferative effect of treatments following APAP induced liver injury, we examined BrdU incorporation during the recovery phase. APAP exposure caused a potent decrease in the number of BrdU positive cells compared to untreated controls and this decrease in proliferation was further blunted by exposure to L-NAME. Embryos treated with GSNORi or NAC demonstrated an increase in the number of BrdU positive cells compared to APAP alone and in combination these compounds synergized to dramatically increase the number of BrdU positive cells in the regenerating liver.

The hepatoprotective effect of GSNORi was also evident in a co-exposure model in which lfabp:GFP embryos were exposed to APAP (5 mM) concurrently with GSNORi and/or NAC from 48-96 hpf. In this model, co-exposure with GSNORi was hepatoprotective and synergized with NAC to further increase liver size. Similarly, the NO donor Deta (5 μM) protected the liver during APAP injury, whereas L-NAME exposure exacerbated injury following APAP exposure. Having found a hepatoprotective effect of GSNORi in embryos exposed to a sublethal dose of APAP, we next examined whether GSNORi was able to enhance survival in embryos exposed to a lethal dose of APAP (10 mM). APAP (10 mM) exposure from 48-96 hpf caused a dramatic loss in survival (4%). Co-exposure with GSNORi (1 μM) or NAC (10 μM) caused a remarkable increase in survival (32%) and the combination treatment increased the survival rate to 49%. In a similar manner, co-exposure with Deta (5 μM) or GSNO (5 μM) increased survival compared to APAP alone. When GSNORi (1 μM) or NAC (10 μM) were given after a 12 hr delay there was a modest increase in survival (18% and 15% respectively), which synergistically increased when treatments were combined (31%). Together, these studies suggest that GSNORi exhibits hepatoprotective activity following APAP injury and enhances survival in zebrafish larvae. Importantly, GSNORi appears to act synergistically with NAC to enhance liver regeneration after APAP injury.

Conserved Hepatoprotective Effects of GSNORi in Adult Zebrafish Exposed to APAP

Having shown that GSNORi was hepatoprotective in APAP exposed larvae, we wanted to examine whether GSNORi remained effective in adult zebrafish. To visualise the hepatoprotective properties of GSNORi in vivo, adult casper lfabp:GFP transgenic fish were exposed to APAP (10 mM) in the presence or absence of GSNORi (1 μM) for 24 hr and examined 72 hpe by fluorescence microscopy. APAP exposure caused a reduction in GFP fluorescence, suggestive of hepatocyte necrosis, whereas co-exposure with the GSNORi prevented the loss in fluorescence. To gain a greater understanding of the hepatoprotective potential of GSNORi, survival studies were conducted in which adults were exposed to APAP (10 mM) in the presence or absence of GSNORi (1 μM) for 24 hr, followed by a 2-day recovery period in fresh water. APAP exposure caused a time-dependent loss in survival reaching 39% by 72 hpe, whereas fish treated with GSNORi (1 μM) performed much better with a markedly enhanced survival rate of 65% by 72 hpe. Similarly, treatment with the NO donors Deta (5 μM) and GSNO (5 μM) enhanced overall survival compared to APAP alone, whereas treatment with L-NAME exacerbated the loss in survival. The differences in survival were reflected in circulating ALT levels, which are used clinically to assess liver toxicity; peaking at 24 hpe with 196 U/L in APAP (10 mM) exposed fish and 55 U/L for fish exposed to APAP with GSNORi (1 μM).

Given that treatments for APAP-induced liver injury are provided after a considerable delay in the clinical setting, we wanted to determine whether GSNORi would remain hepatoprotective when treatment was delayed. In addition, we wanted to ascertain whether GSNORi could synergize with the clinical antidote NAC and extend the therapeutic window. Adult fish were exposed to APAP (10 mM) for 18 hr followed by exposure to GSNORi (1 μM) and/or NAC (10 μM). APAP exposure reduced survival to 57% by 72 hpe, whereas delayed treatment with GSNORi or NAC enhanced survival to 83% and 80% respectively by 72 hpe. Importantly, delayed treatment with the combination of GSNORi and NAC enhanced survival to 95% by 72 hpe. Exposure to APAP (10 mM) caused a dramatic reduction in hepatic glutathione (GSH), which was modulated by exposure to GSNORi (1 μM) and/or NAC (10 μM). To further elucidate the hepatoprotective effects of GSNORi, liver histology was evaluated and this revealed that APAP (10 mM) exposure caused hepatocyte necrosis and hemorrhaging, which was alleviated by exposure to GSNORi (1 μM) and/or NAC (10 μM). Immunohistochemical evaluation of cell proliferation (PCNA) confirmed that GSNORi and NAC modulated liver regeneration following APAP injury. These data suggest that GSNORi is hepatoprotective in the context of APAP-induced liver injury, even after delayed treatment. Importantly, these data also suggest that GSNORi acts synergistically with the clinical antidote NAC to enhance survival.

GSNOR Inhibition Enhances Liver Regeneration After Partial Hepatectomy

In order to determine whether the hepatoprotective and pro-regenerative effect of GSNOR inhibition was limited to APAP-induced liver injury, we determined the impact of exposure to GSNORi after ⅓ partial hepatectomy. This leads to regeneration of hepatic growth as demonstrated in previous assays. Zebrafish underwent liver resection, and assessment of hepatic regrowth at 3 days post resection revealed a 50% increase in liver regrowth after GSNORi exposure (1 μM, 6-18 hours post resection) compared to controls. In contrast, inhibition of NO signaling by L-NAME (10 μM) resulted in complete cessation of regenerative activity. BrdU analysis revealed increased cell proliferation after GSNORi exposure, confirming the pro-proliferative effects of GSNOR inhibition.

GSNOR Deficient Mice Are Protected from APAP Injury

To determine if the effect of GSNOR on APAP-induced liver injury is conserved among vertebrates, we compared the response of WT and GSNOR KO mice. WT and GSNOR KO mice were injected with a sub-lethal dose of APAP (300 mg/kg) and sacrificed for examination at either 6 or 24 hr. Histological examination revealed that WT mice exhibited significant centrilobular hepatocyte necrosis coincident with an increase in TUNEL positive cells, whereas GSNOR KO mice showed a significant decrease in hepatocellular necrosis at both 6 and 24 hr. The circulating ALT values in APAP injected WT mice averaged 1431 U/L at 6 hr and rose to 2584 U/L at 24 hr, whereas GSNOR KO mice had markedly lower ALT values averaging 543 U/L at 6 hr and 1969 U/L at 24 hr. Consequently, GSNOR KO mice exhibit only 38% and 76% of the circulating ALT evident in WT mice at 6hr and 24 hr respectively. These data suggest that GSNOR deficient mice are partially protected from APAP-induced liver injury confirming that GSNOR is a relevant pharmacological target to alleviate liver toxicity following APAP injury.

The Synergistic Hepatoprotective Effects of GSNORi and NAC are Conserved in Mice

The protection that GSNOR deficiency afforded KO mice compared to WT controls suggested that GSNORi could provide hepatoprotection in mice. To investigate this possibility, WT mice were injected with a sub-lethal dose of APAP (300 mg/kg) followed 2 hrs later by treatment with GSNORi (5 mg/kg) and/or NAC (300 mg/kg). Mice were sacrificed at 6 or 24 hr. Histological evaluation demonstrated that GSNORi provided a subtle yet significant level of protection from APAP-induced centrilobular necrosis at both 6 and 24 hr. NAC was effective in preventing liver necrosis, however, the combination therapy of GSNORi and NAC was even more effective with minimal hepatocellular necrosis (TUNEL positive cells) detectable at 24 hr. GSNORi was able to reduce circulating ALT to 72% of the level of APAP alone, while NAC reduced ALT to 43% of the level of APAP alone, whereas combined treatment with GSNORi and NAC caused ALT to decrease to 25% of the level of APAP alone. Together, these experiments suggest that GSNORi acts synergistically with NAC to protect mice from APAP-induced liver injury.

C. List of Conclusions
1. Chemicals affecting NO signaling regulate liver size during development.
2. NO derived from constitutive nos (nos1) is required for optimal liver development.
3. Loss of GSNOR activity prevents S-nitrosothiol metabolism and enhances liver size during development.
4. NO signaling protects zebrafish from APAP-induced injury, even after delayed treatment.
5. Lack of GSNOR activity in GSNOR knockout mice protects from APAP-induced injury
6. Chemical inhibition of GSNOR results in diminished liver damage and enhanced liver regeneration after APAP-injury

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A method of treating a patient at risk for liver damage due to the excessive ingestion of acetaminophen, comprising administering systemically or locally to said patient a therapeutically effective amount of a compound that increases hepatic levels of nitric oxide in said patient.

2. The method of claim 1, wherein said patient at risk for liver damage has ingested more than 2.0 g of acetaminophen within a 24 hour period.

3. The method of claim 1, wherein said patient at risk for liver damage has ingested more than 4.0 g of acetaminophen within a 24 hour period.

4. The method of claim 1, wherein said patient at risk for liver damage has ingested an average of more than 2.0 g of acetaminophen per day for a period of 5 or more consecutive days.

5. The method of claim 1, wherein said patient is treated no later than one week after the ingestion of acetaminophen causing the patient to be at risk for liver damage occurred.

6. The method of claim 1, wherein said patient is exhibiting one or more signs or symptoms associated with liver toxicity, liver damage or hepatic necrosis.

7. The method of claim 1, wherein said compound is an NO donor.

8. The method of claim 1, wherein said compound is selected from the group consisting of: L-arginine, citrulline, ornithine, glutamine, lysine.

9. The method of claim 1, wherein said compound is an S-nitrosothiol.

10. The method of claim 1, wherein said compound is an inhibitor of S-nitrosoglutathione reductase.

11. The method of claim 1, wherein said patient is administered 10 μg to 100 mg of said compound per kg body weight per day for at least one day.

12. The method of claim 1, further comprising administering N-acetylcysteine (Nac) to said patient.

13. The method of claim 1, wherein said compound is administered to said patient by infusion or intravenous injection.

14. The method of claim 1, wherein said compound is administered to said patient orally as part of a pharmaceutical composition in unit dose form.

15. The method of claim 14, wherein said unit dosage form is a tablet or capsule.

16. The method of claim 10, wherein said patient is administered 10 μg to 100 mg of said compound per kg body weight per day for at least one day.

17. The method of claim 16, wherein said patient is treated no later than one week after the ingestion of acetaminophen causing the patient to be at risk for liver damage occurred.

18. The method of claim 17, further comprising administering N-acetylcysteine (Nac) to said patient.

19. The method of claim 17, wherein said compound is administered to said patient by infusion or intravenous injection.

20. The method of claim 17, wherein said compound is administered to said patient orally as part of a pharmaceutical composition in unit dose form.
